(19) United States
(12) Patent Application Publication
(10) Pub. No.: US 2017/0304020 A1
(43) Pub. Date: Oct. 26, 2017

(54) NAVIGATION ARM SYSTEM AND METHODS

(71) Applicants: Samson NG, Toronto (CA); Kyle Richard DUPONT, Toronto (CA); Jakub JANKOWSKI, Toronto (CA); Leila KHERADPIR, Toronto (CA); JeongCheol SEOK, Toronto (CA); Stephen ABELLERA, Toronto (CA)

(72) Inventors: Samson NG, Toronto (CA); Kyle Richard DUPONT, Toronto (CA); Jakub JANKOWSKI, Toronto (CA); Leila KHERADPIR, Toronto (CA); JeongCheol SEOK, Toronto (CA); Stephen ABELLERA, Toronto (CA)

(21) Appl. No.: 15/133,570

(22) Filed: Apr. 20, 2016

(51) Int. Cl. A61B 90/50; A61B 34/30; A61B 17/34; A61B 17/00; A61B 90/00; A61B 34/20

(52) U.S. Cl. CPC A61B 90/50; A61B 90/37; A61B 34/30; A61B 34/20; A61B 17/3423; A61B 17/00234; A61B 2034/2051; A61B 2034/2055; A61B 2560/0437

(57) ABSTRACT

A navigation arm system for supporting a navigation device of a navigation system, capable of coupling with a navigation display cart, the navigation arm system involving: an arm assembly configured to position the navigation device, the arm assembly including a first arm linkage having a tension adjustment feature and releasably connectable to the navigation device; a second arm linkage capable of connecting the arm assembly to the navigation display cart; and at least one joint coupling at least the first and second arm linkages; and a locking mechanism configured to maintain a disposition of the arm assembly in relation to the navigation display cart in a locked stored position and a unlocked deployed position, the locking mechanism configured to lockably couple the arm assembly in relation to the navigation display cart, the locking mechanism including a plurality of locking modes, whereby structural stability is providable

### TECHNICAL FIELD

The present disclosure generally relates to the field of navigation systems. More particularly, the present disclosure technically relates to the field of navigation devices of navigation systems. Even more particularly, the present disclosure technically relates to the field of positioning navigation devices of navigation systems.

### BACKGROUND

In the related art, conventional navigation carts for medical procedures create many hazards in a medical procedure room, such as an operating room, including tripping hazards from cables, tipping hazards from equipment stands or towers, and general hazards associated with clutter because of too many equipment pieces being present in the room.

In addition, challenges experienced in the related art also include inaccuracies involving navigation systems and navigation devices, e.g., the inability to accurately orient a surgical tool having attached passive markers by using an infrared (IR) camera. In the related art, such tracking requires that the surgical tool is disposed within range of the IR camera's measurement volume. Detection of surgical tools tends to fail, depending on an operation room environment, a test set-up, and a patient position, such as a prone or a supine position, etc. The positions of system components are limited by the IR camera position or the location. In the limited space of an operating room, orienting the IR camera in a way that optimizes communication is difficult.

Therefore, a need exists for accurately and stably positioning a navigation device, such as a camera and any other imaging device, in relation to a navigation cart for safe and efficient use in a medical environment, such as an operating room.

### BRIEF SUMMARY

In accordance with embodiments of the present disclosure, a variety of solutions address at least the related art challenges, and involve navigation arm systems and methods that accurately and stably position a navigation device, such as a camera, e.g., an optical camera. In embodiments of the present disclosure, an arm assembly comprises first and second arm linkages having dimensions that increase an imaging volume over that of the related art. The first and second arm linkages facilitate movement control and precise positioning of the navigation device. By using a plurality of arm linkages, the embodiments of the present disclosure provides more degrees of freedom than found in the related art, and facilitates positioning the navigation device within an optimal measurement volume, rather than a restricted surface area, otherwise typical in the related art. The navigation arm systems and methods of the present disclosure also involve a locking feature for maintaining positioning integrity of the navigation device. The navigation arm systems and methods of the present disclosure also restrict movement of the navigation device in two axes, thereby controlling communication between the navigation device and the tracking equipment, and thereby ensuring that the orientation of the tracking features, such as tracking spheres, is correctly tracked.

In accordance with an embodiment of the present disclosure, a navigation arm system for supporting a navigation device of a navigation system, capable of coupling with a navigation display cart, the navigation arm system comprising: an arm assembly configured to position the navigation device, the arm assembly comprising: a first arm linkage having a tension adjustment feature and releasably connectable to the navigation device; a second arm linkage capable of connecting the arm assembly to the navigation display cart; and at least one joint coupling at least the first and second arm linkages; and a locking mechanism configured to maintain a disposition of the arm assembly in relation to the navigation display cart in a locked stored position and a unlocked deployed position, the locking mechanism configured to lockably couple the arm assembly in relation to the navigation display cart, the locking mechanism comprising a plurality of locking modes, whereby structural stability is providable.

In accordance with another embodiment of the present disclosure, a method of fabricating a navigation arm system for supporting a navigation device of a navigation system, capable of coupling with a navigation display cart, the method comprising: providing an arm assembly configured to position the navigation device, providing the arm assembly comprising: providing a first arm linkage having a tension adjustment feature and releasably connectable to the navigation device; providing a second arm linkage capable of connecting the arm assembly to the navigation display cart; and providing at least one joint coupling at least the first and second arm linkages; and providing a locking mechanism configured to maintain a disposition of the arm assembly in relation to the navigation display cart in a locked stored position and a unlocked deployed position, the locking mechanism configured to lockably couple the arm assembly in relation to the navigation display cart, the locking mechanism comprising a plurality of locking modes, whereby structural stability is providable.

In accordance with yet another embodiment of the present disclosure, a method of positioning a navigation device by way of a navigation arm system, comprising: providing the navigation arm system for supporting a navigation device of a navigation system, capable of coupling with a navigation display cart, providing the system comprising: providing an arm assembly configured to position the navigation device, providing the arm assembly comprising:

providing a first arm linkage having a tension adjustment feature and releasably connectable to the navigation device; providing a second arm linkage capable of connecting the arm assembly to the navigation display cart; and providing at least one joint coupling at least the first and second arm linkages; and providing a locking mechanism configured to maintain a disposition of the arm assembly in relation to the navigation display cart in a locked stored position and a unlocked deployed position, the locking mechanism configured to lockably couple the arm assembly in relation to the navigation display cart, the locking mechanism comprising a plurality of locking modes, whereby structural stability is providable; deploying the arm assembly; positioning the navigation device by way of the arm assembly handle; and actuating the locking mechanism, thereby maintaining a disposition of the navigation device.

In an embodiment of the present disclosure, a navigation arm system is configured to operate with a cart, such as a navigation display cart, that accommodates components of a medical navigation system. The cart comprises a frame including a substantially horizontal base having a bottom side and a top side with wheels attached to the bottom side, a substantially vertical column or spine attached to the top side of the base, and optional ballast attached to the base to function as a counterweight to avoid tipping of the cart. The cart may be modular in design and the components of the medical navigation system may be removably attached to the frame. The substantially vertical column is at least partially hollow and comprises a conduit for accommodating cables, wires, and the like. The components of the medical navigation system may include a computing device attached to the frame, the computing device having a processor coupled to a memory and a wireless communication component for communicating wirelessly with a computing device associated with a second cart. The components of the medical navigation system may further include an uninterrupted power supply (UPS) attached to the base and coupled to the computing device for supplying uninterrupted power to the computing device. The cart may further include at least one of a manually-operable arm and a robotic arm attached to an upper end of the substantially vertical column. The cart may further include a display of at least 55 inches in diagonal size mounted on the substantially vertical column, the ballast functioning to ensure stability of the card with the attached display. The cart may further include an arm having a first end and a second end, the first end attached to an upper end of the substantially vertical column, and a tracking camera attached to the second end of the arm such that the tracking camera is positionable above the display.

In an embodiment of the present disclosure, a navigation arm system is configured to operate with a medical navigation system, including a first cart for housing components of the medical navigation system and a second cart for housing components of the medical navigation system. The first cart comprises a frame including a substantially horizontal base having a bottom side and a top side with wheels attached to the bottom side, a substantially vertical column attached to the top side of the base, and a ballast attached to the base to function as a counterweight to avoid tipping of the cart. The components of the medical navigation system include a computing device attached to the frame, the computing device having a processor coupled to a memory and a wireless communication component for communicating wirelessly with a computing device associated with the second cart. The second cart may be for housing further components of the medical navigation system. The second cart comprises a second frame including a second substantially horizontal base having a bottom side and a top side with wheels attached to the bottom side, a second substantially vertical column attached to the top side of the base, a second ballast attached to the base to function as a counterweight to avoid tipping of the cart; and a display of at least 55 inches in diagonal size mounted on the second substantially vertical column.

Benefits of the systems and methods of the present disclosure include, but are not limited to, increasing the measurement volume by way of a larger range of positioning relative to that of the related art IR cameras, whereby a navigation device, such as an optical camera, is also optimally disposable in relation to a navigation display device disposed on a navigation display cart, by way of a navigation arm system, whereby the navigation device is optimally disposable, storable, and transportable in relation to a user, such as a neurosurgeon, and whereby resolution of tracking markers, e.g., tracking spheres and equipment, e.g., in use by the neurosurgeon, is maintained.

Some of the features in the present disclosure are broadly outlined in order that the section, entitled Detailed Description, is better understood and that the present contribution to the art is better appreciated. Additional features of the present disclosure are described hereinafter. In this respect, understood is that the present disclosure is not limited in its application to the details of the components or steps set forth herein or as illustrated in the several figures of the being carried out in various ways. Also, understood is that the phraseology and terminology employed herein are for the purpose of the description and should not be regarded as limiting.

### BRIEF DESCRIPTION OF THE DRAWING

The above, and other, aspects, features, and benefits of several embodiments of the present disclosure will be more apparent from the following Detailed Description as presented in conjunction with the following several figures of the Drawing.

FIG. 1 is a diagram illustrating an access port inserted into a human brain for providing access to internal brain tissue during a medical procedure, in accordance with an embodiment of the present disclosure.

FIG. 2 is a diagram illustrating an exemplary navigation system for supporting minimally invasive access port-based surgery, in accordance with an embodiment of the present disclosure.

FIG. 3 is a block diagram illustrating a control and processing system operable with the navigation system, as shown in FIG. 2, in accordance with an embodiment of the present disclosure.

FIG. 4A is a flow diagram illustrating a method of conducting a surgical procedure by using the navigation system, as shown in FIG. 2, in accordance with an embodiment of the present disclosure.

FIG. 4B is a flow diagram illustrating a method of registering a patient for conducting a surgical procedure, as shown in FIG. 4A, by using the navigation system, as shown in FIG. 2, in accordance with an embodiment of the present disclosure.

FIG. 5 is a diagram illustrating a perspective view of a cart for housing components of a medical navigation system, in accordance with an embodiment of the present disclosure.

FIG. 6 is a diagram illustrating a perspective view of a cart for housing components of a medical navigation system, in accordance with an alternative embodiment of the present disclosure.

FIG. 7A is a diagram illustrating a perspective view showing a cart for housing components of a medical navigation system, in accordance with an alternative embodiment of the present disclosure.

FIG. 7B is a diagram illustrating a front view of the cart, as shown in FIG. 7A, in accordance with an embodiment of the present disclosure.

FIG. 8A is a diagram illustrating a perspective view of a cart for housing components of a medical navigation system, in accordance with an alternative embodiment of the present disclosure.

FIG. 8B is a diagram illustrating a side view of the cart, as shown in FIG. 8A, in accordance with an embodiment of the present disclosure.

FIG. 9 is a diagram illustrating a side view of the cart, as shown in FIG. 8A, with additional components, in accordance with an embodiment of the present disclosure.

FIG. 10 is a diagram illustrating a side view of a modification to the cart, as shown in FIG. 8A, in accordance with an embodiment of the present disclosure.

FIG. 11 is a diagram illustrating a perspective view of a navigation arm system, as undeployed or stored, operable with a navigation display cart and in relation to a navigation display device of a navigation system, in accordance with an embodiment of the present disclosure.

FIG. 12 is a diagram illustrating a front view of a navigation arm system, as undeployed or stored, operable with a navigation display cart and in relation to a navigation display device of a navigation system, in accordance with an embodiment of the present disclosure.

FIG. 13 is a diagram illustrating a side view of a navigation arm system, as undeployed or stored, operable with a navigation display cart and in relation to a navigation display device of a navigation system, in accordance with an embodiment of the present disclosure.

FIG. 14 is a diagram illustrating a perspective view of a navigation arm system, as deployed, operable with a navigation display cart and in relation to a navigation display device of a navigation system, in accordance with an embodiment of the present disclosure.

FIG. 15 is a diagram illustrating a front view of a navigation arm system, as deployed, operable with a navigation display cart and in relation to a navigation display device of a navigation system, in accordance with an embodiment of the present disclosure.

FIG. 16 is a diagram illustrating a side view of a navigation arm system, as deployed, operable with a navigation display cart and in relation to a navigation display device of a navigation system, in accordance with an embodiment of the present disclosure.

FIG. 17A is a diagram illustrating a front view of a navigation arm system, in the undeployed or stored disposition, as shown FIG. 11, in accordance with an embodiment of the present disclosure.

FIG. 17B is a diagram illustrating a side view of a navigation arm system, in the undeployed or stored disposition, as shown FIG. 11, in accordance with an embodiment of the present disclosure.

FIG. 17C is a diagram illustrating an opposing side view of a navigation arm system, in the undeployed or stored disposition, as shown FIG. 11, in accordance with an embodiment of the present disclosure.

FIG. 18 is a diagram illustrating a perspective view of a navigation arm system, in the deployed disposition, as shown FIG. 14, in accordance with an embodiment of the present disclosure.

FIG. 19A is a diagram illustrating a top view of a navigation arm system, in the undeployed or stored disposition, as shown FIG. 11, in accordance with an embodiment of the present disclosure.

FIG. 19B is a diagram illustrating a rear perspective view of a navigation arm system, in the undeployed or stored disposition, as shown FIG. 11, in accordance with an embodiment of the present disclosure.

FIG. 19C is a diagram illustrating a rear perspective view of a navigation arm system, in the undeployed or stored disposition, as shown FIG. 11, in accordance with an embodiment of the present disclosure.

FIG. 19D is a diagram illustrating a side view of a navigation arm system, in the undeployed or stored disposition, as shown FIG. 11, in accordance with an embodiment of the present disclosure.

FIG. 19E is a diagram illustrating a rear view of a navigation arm system, in the undeployed or stored disposition, as shown FIG. 11, in accordance with an embodiment of the present disclosure.

FIG. 19F is a diagram illustrating a front view of a navigation arm system, in the undeployed or stored disposition, as shown FIG. 11, in accordance with an embodiment of the present disclosure.

FIG. 20 is a diagram illustrating an exploded perspective view of a navigation arm system, in the undeployed or stored disposition, as shown FIG. 11, in accordance with an embodiment of the present disclosure.

FIG. 20A is a diagram illustrating an exploded perspective view of a second arm linkage, or rigid arm, of an arm assembly in a navigation arm system, as shown FIG. 20, in accordance with an embodiment of the present disclosure.

FIG. 20B is a diagram illustrating an exploded perspective view of an extension arm stem in a navigation arm system, as shown FIG. 20, in accordance with an embodiment of the present disclosure.

FIG. 20C is a diagram illustrating an exploded perspective view of arm joint stem of a navigation arm system, as shown FIG. 20, in accordance with an embodiment of the present disclosure.

FIG. 20D is a diagram illustrating a perspective view of a first arm linkage, or a spring arm, of an arm assembly in a navigation arm system, as shown FIG. 20, in accordance with an embodiment of the present disclosure.

FIG. 20E is a diagram illustrating a exploded cut-away perspective view of a first arm linkage, or a spring arm, of an arm assembly in a navigation arm system, as shown FIG. 20, in accordance with an embodiment of the present disclosure.

FIG. 20F is a diagram illustrating a perspective view of a first arm linkage, or a spring arm, further comprising a cover member, of an arm assembly in a navigation arm system, as shown FIG. 20, in an undeployed position, in accordance with an embodiment of the present disclosure.

FIG. 20G is a diagram illustrating a perspective view of an arm assembly handle of an arm assembly in a navigation arm system, as shown FIG. 20, in accordance with an embodiment of the present disclosure.

FIG. 20H is a diagram illustrating a perspective view of a navigation device mounting joint of an arm assembly of navigation arm system, as shown FIG. 20, in accordance with an embodiment of the present disclosure.

FIG. 21 is a diagram illustrating an exploded perspective view of a mounting bracket and locking mechanism of a navigation arm system, in accordance with an embodiment of the present disclosure.

FIG. 22 is a diagram illustrating an exploded perspective view of a joint for rotatably coupling first and second arm linkages of an arm assembly in a navigation arm system, in accordance with an embodiment of the present disclosure.

FIG. 23A is a diagram illustrating an exploded perspective view of a navigation device mounting joint for rotatably coupling a first arm linkage, of an arm assembly in a navigation arm system, with a navigation device, in accordance with an embodiment of the present disclosure.

FIG. 23B is a diagram illustrating a perspective view of a navigation device mounting joint for rotatably coupling a first arm linkage, of an arm assembly in a navigation arm system, with a navigation device, in accordance with an embodiment of the present disclosure.

FIG. 24A is a diagram illustrating a perspective view of a locking mechanism in a navigation arm system, in accordance with an embodiment of the present disclosure.

FIG. 24B is a diagram illustrating an exploded perspective view of a locking mechanism in a navigation arm system, in accordance with an embodiment of the present disclosure.

FIG. 25A is a diagram illustrating a top view of a ring handle in a locking mechanism, disposed in a unlocked position, in accordance with an embodiment of the present disclosure.

FIG. 25B is a diagram illustrating a top view of a ring handle in a locking mechanism, disposed in an locked position, in accordance with an embodiment of the present disclosure.

FIG. 26A is a diagram illustrating a bottom view of a ring handle in a locking mechanism, disposed in a unlocked position, in accordance with an embodiment of the present disclosure.

FIG. 26B is a diagram illustrating a bottom view of a ring handle in a locking mechanism, disposed in an locked position, in accordance with an embodiment of the present disclosure.

FIG. 27A is a diagram illustrating a cut-away perspective view of a ring lock in a locking mechanism L interacting with a handle portion of an arm assembly handle, disposed in a fully unlocked position, in accordance with an embodiment of the present disclosure.

FIG. 27B is a diagram illustrating a cut-away perspective view of a ring handle in a locking mechanism L interacting with a handle portion of an arm assembly handle, disposed in a partially locked position, in accordance with an embodiment of the present disclosure.

FIG. 27C is a diagram illustrating a cut-away perspective view of a ring handle in a locking mechanism L interacting with a handle portion of an arm assembly handle, disposed in a fully locked position, in accordance with an embodiment of the present disclosure.

FIG. 28A is a diagram illustrating a cut-away perspective view of a ring lock in a locking mechanism interacting with a handle portion of an arm assembly handle, disposed in a fully unlocked position and ready to accept the handle portion of an arm assembly handle, in accordance with an embodiment of the present disclosure.

FIG. 28B is a diagram illustrating a cut-away perspective view of a ring lock in a locking mechanism interacting with a handle portion of an arm assembly handle, disposed in a fully locked position, having accepted the handle portion of an arm assembly handle, in accordance with an embodiment of the present disclosure.

FIG. 29A is a diagram illustrating a side view of the locking mechanism interacting with a navigation device handle, disposed in a fully locked position, having accepted the navigation device handle, in accordance with an embodiment of the present disclosure.

FIG. 29B is a diagram illustrating a close side view of the locking mechanism interacting with a navigation device handle, disposed in a fully locked position, having accepted the navigation device handle, wherein the navigation device mounting joint is configured to make a hard stop for an angular disposition of approximately 180 degrees, in accordance with an embodiment of the present disclosure.

FIG. 30 is a diagram illustrating a perspective view of a navigation device handle, as comprising a plurality of notches for engaging the cams of the ring handle, in accordance with an embodiment of the present disclosure.

FIG. 31A is a diagram illustrating a perspective view of a locking mechanism, comprising a ring handle, interacting with a navigation device handle, disposed in a fully unlocked position and ready to accept the navigation device handle, in accordance with an embodiment of the present disclosure.

FIG. 31B is a diagram illustrating a perspective view of a locking mechanism, comprising a ring handle, interacting with a navigation device handle, disposed in a fully locked position, having accepted the navigation device handle, in accordance with an embodiment of the present disclosure.

FIG. 32 is a flow diagram illustrating a method of fabricating a navigation arm system, in accordance with an embodiment of the present disclosure

FIG. 33 is a flow diagram illustrating a method of positioning a navigation device by way of a navigation arm system, in accordance with an embodiment of the present disclosure.

Corresponding reference numerals or characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in commercially feasible embodiment are often not depicted in order to facilitate a less obstructed view of these various embodiments of the present disclosure.

### DETAILED DESCRIPTION

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves. As used herein, the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port. The present disclosure is generally related to image guided medical procedures using a surgical instrument, such as a fibre optic scope, an optical coherence tomography (OCT) probe, a micro ultrasound transducer, an electronic sensor or stimulator, or an access port based surgery.

In the example of a port-based surgery, a surgeon or robotic surgical system may perform a surgical procedure involving tumor resection in which the residual tumor remaining after is minimized, while also minimizing the trauma to the intact white and grey matter of the brain. In such procedures, trauma may occur, for example, due to contact with the access port, stress to the brain matter, unintentional impact with surgical devices, and/or accidental resection of healthy tissue. A key to minimizing trauma is having the appropriate tools and equipment for use by a surgeon so that the medical procedure can be performed quickly, accurately, and safely.

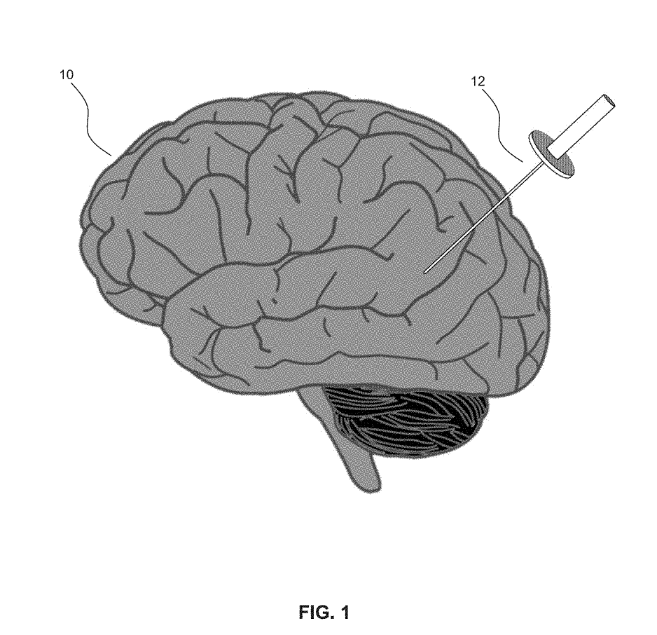

Referring to FIG. 1, this diagram illustrates, in a perspective view, an access port 12 being inserted into a human brain 10 for providing access to internal brain tissue during a medical procedure, in accordance with an embodiment of the present disclosure. The access port 12 is inserted into a human brain 10, providing access to internal brain tissue. Access port 12 may include such instruments as catheters, surgical probes, or cylindrical ports such as the NICO® BrainPath®. Surgical tools and instruments may then be inserted within the lumen of the access port in order to perform surgical, diagnostic or therapeutic procedures, such as resecting tumors as necessary. The present disclosure applies equally well to catheters, DBS needles, a biopsy procedure, and also to biopsies and/or catheters in other medical procedures performed on other parts of the body. In the example of a port-based surgery, a straight or linear access port 12 is typically guided down a sulci path of the brain. Surgical instruments would then be inserted down the access port 12.

Still referring to FIG. 1, optical tracking systems, used in the medical procedure, track the position of a part of the instrument that is within line-of-site of the optical tracking camera. These optical tracking systems also require a reference to the patient to know where the instrument is relative to the target, e.g., a tumor, of the medical procedure. These optical tracking systems require a knowledge of the dimensions of the instrument being tracked so that, for example, the optical tracking system knows the position in space of a tip of a medical instrument relative to the tracking markers being tracked. All of this requires the appropriate computer based equipment to be provided at the site of the medical procedure in a form that is easy for the surgical team to use and does not create any hazards in the room where the medical procedure will be performed.

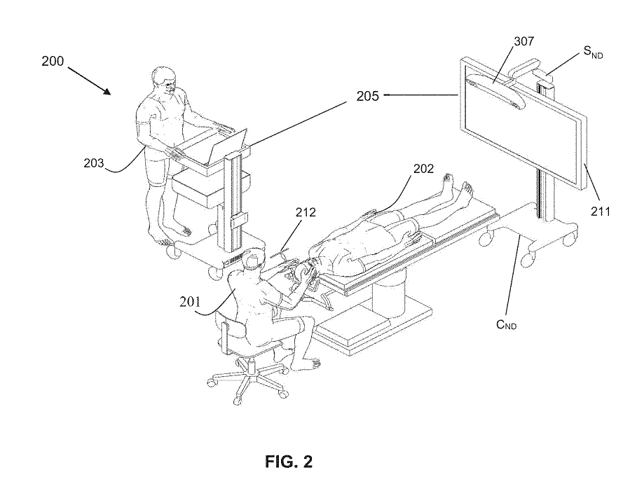

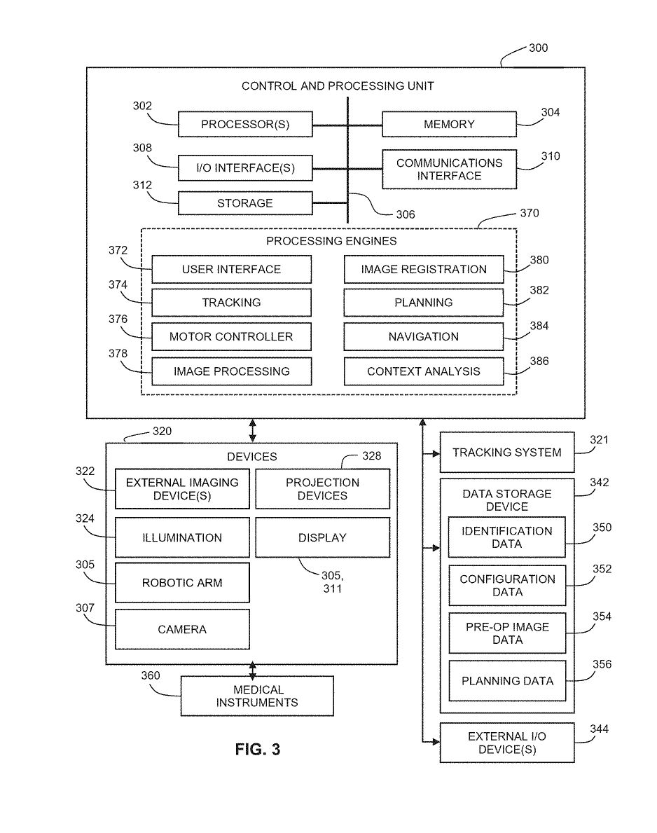

Referring to FIG. 2, this diagram illustrates an exemplary medical navigation system 205 for supporting minimally invasive access port-based surgery, in accordance with an embodiment of the present disclosure. An exemplary navigation system environment 200 is shown, which may be used to support navigated image-guided surgery. A surgeon 201 conducts a surgery on a patient 202 in an operating room (OR) environment. A medical navigation system 205 comprising an equipment tower (not shown), a tracking system 321 (FIG. 3), displays 211 and tracked instruments 212 assist the surgeon 201 during his procedure. An operator 203 is also present to operate, control and provide assistance for the medical navigation system 205.

Referring to FIG. 3, this block diagram illustrates a control and processing system 300 operable with the navigation system 205, as shown in FIG. 2, in accordance with an embodiment of the present disclosure. The control and processing system 300 is operable in the medical navigation system 200 and may be part of the equipment tower. In one example, the control and processing system 300 comprises one or more processors 302, a memory 304, a system bus 306, one or more input/output interfaces 308, a communications interface 310, and storage device 312. The control and processing system 300 is interfaceable with other external devices, such as tracking system 321, a data storage device 342, and external user input and output devices 344, which may include, for example, one or more of a display, keyboard, mouse, sensors attached to medical equipment, foot pedal, and microphone and speaker. The data storage device 342 may be any suitable data storage device, such as a local or remote computing device, e.g., a computer, hard drive, digital media device, or server) having a database stored thereon. The data storage device 342 comprises identification data 350 for identifying one or more medical instruments 360 and configuration data 352 that associates customized configuration parameters with one or more medical instruments 360. The data storage device 342 further comprises preoperative image data 354 and/or medical procedure planning data 356. Although the data storage device 342 is shown as a single device, understood is that, in other embodiments, the data storage device 342 comprises multiple storage devices and is also encompassed by the present disclosure.

Still referring to FIG. 3, the medical instruments 360 are identifiable by the control and processing unit 300. The medical instruments 360 are connectable with, and controllable by the control and processing unit 300. Alternatively, the medical instruments 360 are operable, or otherwise employable, independent of the control and processing unit 300. The tracking system 321 is employable for tracking one or more of the medical instruments 360 and for spatially registering the one or more tracked medical instruments to an intraoperative reference frame. For example, the medical instruments 360 comprise tracking markers, such as tracking spheres recognizable by a tracking camera 307. In one example, the tracking camera 307 comprises an infrared (IR) tracking camera. In alternate embodiments, the tracking camera 307 comprises an optical camera or a stereoscopic camera, capable of supporting 3D images or holograms. In another example, a sheath placed over a medical instrument 360 is connectable to, and controllable by the control and processing unit 300.

Still referring to FIG. 3, the control and processing unit 300 is also interfaceable with a number of configurable devices 320, and adapted to intraoperatively reconfigure one or more of such configurable devices based on configuration parameters obtained from the configuration data 352. Examples of the configurable devices 320 include one or more external imaging devices 322, one or more illumination devices 324, a robotic arm, one or more projection devices 328, and one or more displays 205, 211.

Still referring to FIG. 3, exemplary aspects of the present disclosure are implementable via at least one of the processor(s) 302 and the memory 304. For example, the functionalities, described herein, are at least partially implementable, via hardware logic in the processor 302 and via instructions stored in the memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately, in one example, the processing modules 370 are storable in the memory 304; and the processing modules are collectively denoted as processing modules 370. Understood is that the system 205 is not intended to be limited to the components as shown. One or more components of the control and processing system 300 are providable as an external component or device. In one example, the navigation module 384 is providable as an external navigation system that is integrable with the control and processing system 300.

Still referring to FIG. 3, some embodiments may be implemented using processor 302 without additional instructions stored in memory 304. Some embodiments may be implemented using the instructions stored in memory 304 for execution by one or more general purpose microprocessors. Thus, the present disclosure is not limited to a specific configuration of hardware and/or software. While some embodiments can be implemented in fully functioning computers and computer systems, various embodiments are capable of being distributed as a computing product in a variety of forms and are capable of being applied regardless of the particular type of machine or computer readable media used to actually effect the distribution.

Still referring to FIG. 3, at least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device. A computer readable storage medium can be used to store software and data which, when executed by a data processing system, causes the system to perform various methods. The executable software and data may be stored in various places including for example ROM, volatile RAM, non-volatile memory and/or cache. Portions of this software and/or data may be stored in any one of these storage devices.

Still referring to FIG. 3, examples of computer-readable storage media include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions may be embodied in digital and analog communication links for electrical, optical, acoustical or other forms of propagated signals, such as carrier waves, infrared signals, digital signals, and the like. The storage medium may be the Internet cloud, or a computer readable storage medium such as a disc.

Still referring to FIG. 3, at least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

Still referring to FIG. 3, according to one aspect of the present disclosure, a feature of the navigation system 205, comprising the control and processing unit 300, includes providing tools to the neurosurgeon, e.g., the surgeon 201, that will lead to the most informed and the least damaging neurosurgical procedures. In addition to removal of brain tumours and intracranial hemorrhages (ICH), the navigation system 205 is applicable to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

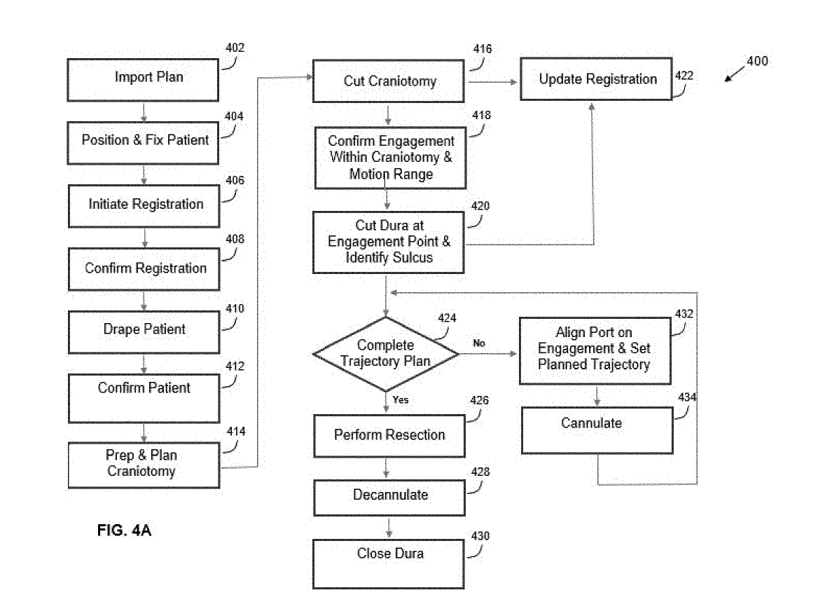

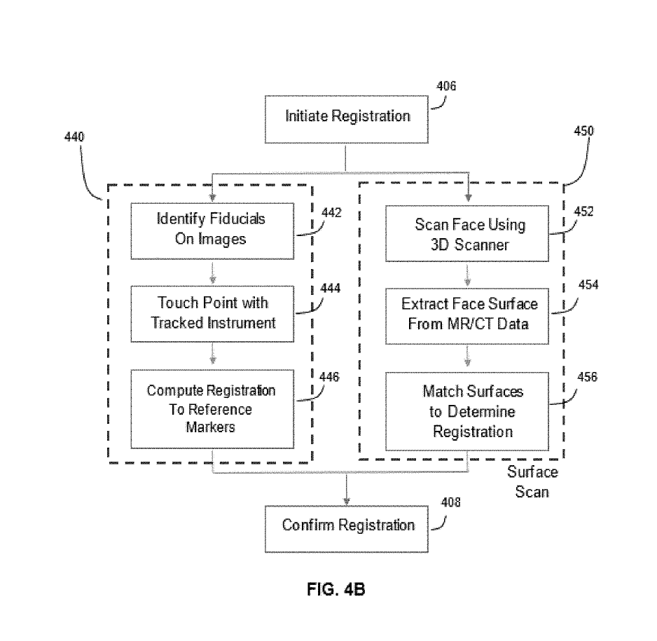

Referring to FIG. 4A, this flow diagram illustrates a method 400 of conducting a surgical procedure by using the navigation system, as shown in FIG. 2, in accordance with an embodiment of the present disclosure. At a first block 402, the step of importing a port-based surgical plan is performed. A detailed description of the steps for creating and selecting a surgical plan is outlined in the following priority documents, as described in the section Cross-Reference to Related Applications(s), and are encompassed by the present disclosure: PCT Publication No. WO/2014/139024, entitled “PLANNING, NAVIGATION AND SIMULATION SYSTEMS AND METHODS FOR MINIMALLY INVASIVE THERAPY,” U.S. Provisional Patent Application Ser. Nos. 61/800,155 and 61/924,993.

Still referring to FIG. 4A, after importing the surgical plan into the navigation system 205, as indicated by block 402, the patient 202 is affixed into position using a body holding mechanism. The head position is also confirmed with the patient plan in the navigation system 205 (block 404), which, in one example, is implementable by the computer or controller forming part of the equipment tower 211. Next, registration of the patient 202 is initiated (block 406). The phrase “registration” or “image registration” refers to the process of transforming different sets of data into one coordinate system. Data comprises multiple photographs, data from different sensors, times, depths, or viewpoints. The process of “registration” is used in the present disclosure for medical imaging in which images from different imaging modalities (multi-modal) are co-registered. Registration is used in order to be able to compare or integrate the data obtained from these different modalities.

Still referring to FIG. 4A, appreciated is that numerous registration techniques are available and one or more of the techniques may be applied and are encompassed by the present disclosure. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

Referring now to FIG. 4B, this flow diagram illustrates the step of registering a patient for conducting a surgical procedure, as indicated by block 406, in the method 400, as shown in FIG. 4A, by using the navigation system 205, as shown in FIG. 2, in accordance with an embodiment of the present disclosure. If the use of fiducial touch points, as indicated by block 440, is contemplated, the step 406 of method 400 comprises first identifying fiducial markers on images, as indicated by block 442, then touching the touch points with a tracked instrument, as indicated by block 444. Next, the navigation system 205 computes the registration to reference markers, as indicated by block 446. Alternately, registration can also be completed by conducting a surface scan procedure, as indicated by block 450. The block 450 represents an alternative approach, but is not required when using a fiducial pointer. First, the face is scanned using a 3D scanner, as indicated by block 452. Next, the face surface is extracted from MR/CT data, as indicated by block 454. Finally, surfaces are matched to determine registration data points, as indicated by block 456. Upon completion of either the fiducial “touch points” step, as indicated by block 440, or the surface scan step, as indicated by block 450, the data extracted is computed and used to confirm registration, as indicated by block 408.

Still referring to FIG. 4B and referring back to FIG. 4A, once registration is confirmed, as indicated by block 408, the patient is draped, as indicated by block 410. Typically, draping involves covering the patient and surrounding areas with a sterile barrier to create and maintain a sterile field during the surgical procedure. The purpose of draping is to eliminate the passage of microorganisms, e.g., bacteria, between non-sterile and sterile areas. At this point, the non-sterile patient reference is replaced with a sterile patient reference of identical geometry location and orientation. Numerous mechanical methods may be used to minimize the displacement of the new sterile patient reference relative to the non-sterile one that was used for registration, but inevitable is that some error will exist. This error directly translates into registration error between the surgical field and pre-surgical images. In fact, the farther away points of interest are from the patient reference, the worse the error will be. The navigation system 205 provides a solution to minimizing such error. Upon completion of draping, as indicated by block 410, the patient engagement points are confirmed, as indicated by block 412, and then the craniotomy is prepared and planned, as indicated by block 414. Upon completion of the preparation and planning of the craniotomy, as indicated by block 414, the craniotomy is performed, i.e., the cranium is cut and a bone flap is temporarily removed from the skull to access the brain, as indicated by block 416. Registration data is updated with the navigation system at this point, as indicated by block 422.

Referring back to FIG. 4A, next, the engagement within craniotomy and the motion range are confirmed, as indicated by block 418. Next, the procedure advances to cutting the dura at the engagement points and identifying the sulcus, as indicated by block 420. Thereafter, the cannulation process is initiated, as indicated by block 424. Cannulation involves inserting a port, such as the access port 12, into the brain 10, typically along a sulci path, as indicated by block 420, along a trajectory plan. Cannulation is typically an iterative process that involves repeating the steps of aligning the port on engagement and setting the planned trajectory, as indicated by block 432, and then cannulating to the target depth, as indicated by block 434, until the complete trajectory plan is executed, as indicated by block 424. Once cannulation is complete, the surgeon 201 then performs resection, as indicated by block 426, to remove part of the brain 10 and/or tumor of interest. The surgeon 201 then decannulates, as indicated by block 428, by removing the port and any tracking instruments from the brain. Finally, the surgeon closes the dura and completes the craniotomy, as indicated by block 430. Some aspects of the present disclosure that are shown in FIG. 4A are specific to port-based surgery, such as indicated by blocks 428, 420, and 434, but the appropriate portions of these steps are optional or are suitably modified when performing non-port based surgery and are also encompassed by the present disclosure.

Referring back to FIGS. 4A and 4B, when performing a surgical procedure, the medical navigation system 205 acquires and maintains a reference of the location of the tools in use as well as the patient in three dimensional (3D) space. In other words, during a navigated neurosurgery, a tracked reference frame that is fixed relative to the patient's skull is useful. During the registration phase of a navigated neurosurgery, as indicated by block 406, a transformation is calculated that maps the frame of reference of preoperative MRI or CT imagery to the physical space of the surgery, specifically to the patient's head. Calculating the transformation is accomplished by the navigation system 205 tracking locations of fiducial markers fixed to the patient's head, relative to the static patient reference frame. The patient reference frame is typically rigidly attached to the head fixation device, such as a Mayfield clamp. Registration is typically performed before the sterile field has been established, as indicated by block 410.

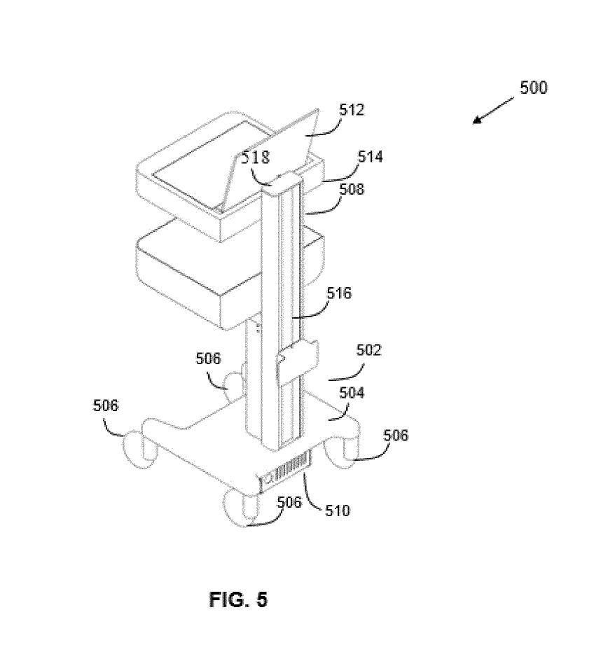

Referring to FIG. 5, this diagram illustrates, in a perspective view, a cart 500 for housing components (not shown) of a medical navigation system 205, in accordance with an embodiment of the present disclosure. The navigation system, such as the medical navigation system 205 (FIG. 2), comprises the control and processing system 300 (FIG. 3). The cart 500 includes a frame 502 comprising a base 504 having a bottom side and a top side. In one example, the base is substantially horizontal and may have wheels 506 attached to the bottom side of the base. The frame 502 may further include a column 508 attached to the top side of the base. In one example, the column 508 may be substantially vertical. The frame 502 may further include a ballast 510 attached to the base, where the ballast may function as a counterweight to avoid tipping of the cart 500.

Still referring to FIG. 5, in one example, the ballast 510 may be attached to the bottom side of the base 504 between the wheels 506. The base 504 may have four corners and the four wheels 506 may be configured such that each wheel 506 is attached near a corner of the base 504. While the ballast 510 is shown attached to the bottom side of the base 504, the ballast 510 may also be attached to the top side of the base 504, or any other suitable location to meet the design criteria of a particular application. Likewise, while the example in FIG. 5 shows four wheels 506, three wheels, five wheels, six wheels, or any other suitable number of wheels may be used to meet the design criteria of a particular application, where an objective may include maximizing or achieving substantial stability of the cart 500.

Still referring to FIG. 5, the components of the medical navigation system 205 housed on the cart 500 may include a computing device 512 attached to the frame 502. The computing device 512 includes a portable computer such as a laptop computer resting on a shelf 514 that is attached to the column 508. In one example, the shelf 514 may be foldable relative to the column 508, such as by a hinge mechanism attaching the shelf 514 to the column 508. The computing device 512 may also be considered part of the control and central processing unit 300 of navigation system 205. The computing device 512 may have a processor, e.g., the processor 302, coupled to a memory, e.g., memory 304, and a wireless communication component, e.g., communications interface 310, for communicating wirelessly with a computing device associated with a second cart, such as the cart 600 discussed below in connection with FIG. 6. The wireless communications components 310 may include short and long range protocols such as Bluetooth, Zigbee, IRDA, Wi-Fi, GSM, CDMA, LTE or any other suitable existing or yet to be developed wireless communications protocol.

Still referring to FIG. 5, the components of the medical navigation system 205 further comprise an uninterrupted power supply (UPS) attached to the base 504 and coupled to the computing device 512 for supplying uninterrupted power to the computing device 512. The UPS is disposable in the same position as the ballast 510. Since a UPS is typically heavy, the UPS may form part of the ballast 510 or may be located next to the ballast 510 to help stabilize the cart 500. In another example, the UPS may be located on top of the base 504. While some examples for the location of the UPS have been provided, the UPS may be located in any suitable location on the cart 500 to meet the design criteria of a particular application.

Still referring to FIG. 5, in one example, the column 508 may be at least partially hollow therefore including a conduit for cables and power management. The column 508 may be used for cable management for concealing cables, e.g., power cord, or networking, USB, audio, video cables, connecting computing device 512 to a UPS, external hard drives, CDROMs, or wireless communications component 310 on navigation cart 500 and to other components of navigation system 205 such as display 602 and/or tracking camera 610 on auxiliary cart 600, as seen in FIG. 6. The column 508 may be a fixed length or may also be extendible, such as being a modular design where smaller subsections of the column 508 may be attached together to create a needed length of the column 508. Vertical extension of column 508 may be raised (and also lowered) by a release mechanism either manually or automatically (i.e., motorized). The hollow conduit of column 508 may also house a power bar or electrical extension cord.

Still referring to FIG. 5, a back cover 516 is shown attached to the column 508 to retain and hide cables placed within the column 508. An end cap 518 is also shown placed on the top end of the column 508. In the situation where a longer column is desired, the end cap 518 may be removed and additional sections of the column 508 may be attached to the top end of the column 508. End cap 518 may be removed and replaced by a connecting joint (not shown) that connects the top of column 508 to a robotic arm, tracking camera, imaging camera or other accessories used by the navigation system 205.

Referring now to FIG. 6, this diagram illustrates, in a perspective view, a cart, e.g., an auxiliary cart 600, for housing components of a medical navigation system 205, in accordance with an alternative embodiment of the present disclosure. The auxiliary cart 600 houses additional components of the medical navigation system 205. In one example, the auxiliary cart 600 may comprise at least some of the features of the cart 500 (FIG. 5). The carts 500 and 600 are modular and may share many of the same components. In this regard, like components are shown with like reference numerals, wherein the auxiliary cart 600 further comprises the frame 502, the base 504, the wheels 506, the column 508, and the ballast 510 (not shown in

FIG. 6). Auxiliary cart 600 comprises a column 508 that is taller than cart 500, which may be constructed using two or more like columnar portions attached together. However, in another example columnar portions may be available in different lengths and column 508 of cart 600 may be comprised of only one columnar portion. In the example shown in FIG. 6, auxiliary cart 600 also include a display 602 mounted on the column 508. In one example, the display 602 may be at least approximately 55 inches in diagonal size, therefore providing a superior viewing experience for the surgeon as opposed to conventional solutions. In one example, the display 602 is mounted on a front side of the column 508. The ballast 510 may be designed to ensure stability of the card with the attached display 602, for example by ensuring that ballast 510 is heavy enough to prevent tipping of auxiliary cart 600 with the display 602 attached.

Still referring now to FIG. 6, the auxiliary cart 600 may also include an arm 604 having a first end 606 and a second end 608, where the first end 606 is attached to an upper end of the column 508 and the second end 608 is attached to a tracking camera 610, e.g., the camera 307 and/or the tracking system 321, such that the tracking camera 610 is positionable above the display 602. The components of the medical navigation system housed on the auxiliary cart 600 comprise a computing device 512 (not shown) attached to the frame 502. The computing device 512 is integrable into the display 602 or attached behind the display 602. The computing device 512 comprises a processor, e.g., processor 302, coupled to a memory, e.g., memory 304, and a wireless communication component, e.g., communications interface 310, for communicating wirelessly with a computing device associated with the first cart 500. The wireless communications components 310 may include Bluetooth, Zigbee, IRDA, Wi-Fi, GSM, CDMA, LTE or any other suitable existing or yet to be developed wireless communications protocol. In this way, the display 602 and/or tracking camera 610 may communicate wirelessly with the computing device 512 of FIG. 5 such that no cables need to be placed across the operating room floor, thereby eliminating a tripping hazard. The components of the medical navigation system included in FIG. 6 may further include an uninterrupted power supply (UPS) attached to the base 504 and coupled to the display 602 for supplying uninterrupted power to the display 602. In the example shown in FIG. 6, the UPS may be located in the same position as the ballast 510, e.g., on the bottom of the base 504.

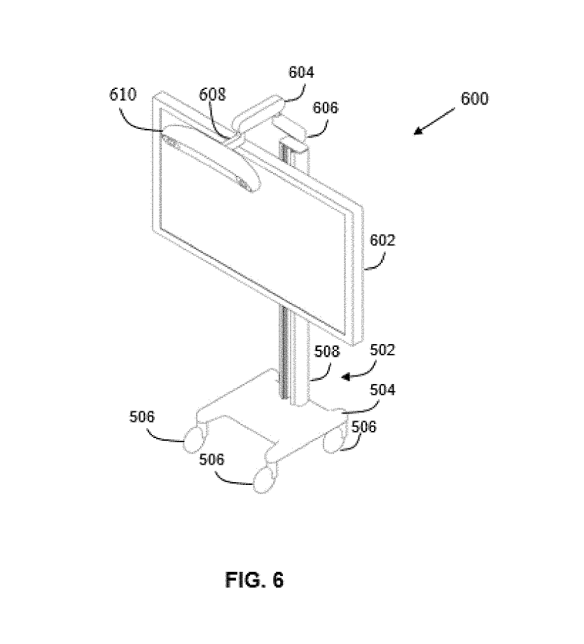

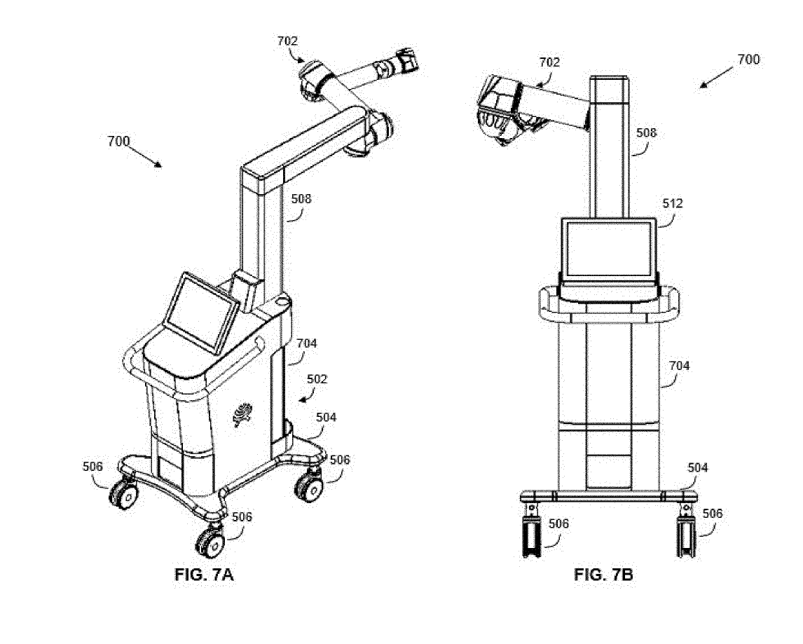

Referring to FIG. 7A, this diagram illustrates, in a perspective view, a cart 700 for housing components of a medical navigation system 205, in accordance with an alternative embodiment of the present disclosure. In one example, the cart 700 comprises at least some of the features of the cart 500 and is modular. In this regard, like components are shown with like reference numerals, wherein the cart 700 also comprises the frame 502, the base 504, the wheels 506, the column 508, the ballast 510 (not shown), and the computing device 512. The ballast 510 may be placed on the top side of the base 504. The cart 700 may have a column 508 that is taller than the cart 500, the column 508 comprising two or more like columnar portions attached together. However, in another example, the columnar portions each comprise a distinct length; and the column 508 of the cart 700 comprises only one columnar portion.

Still referring to FIG. 7A, the cart 700 may include a robotic arm 702 attached to an upper end of the column 508. The end effector of the robotic arm 702 is attachable to an optical camera, such as the camera 307, or a surgical microscope (not shown), providing enhanced images, e.g., picture or video, of the surgical procedure, which may be displayed on display 602. Optionally, the end effector of robotic arm 702 may also be equipped with alternate imaging modality devices, i.e., MRI probe, Raman spectroscopy probe, ultrasound probe) which may provide intraoperative multi-modal reading of information for the surgical procedure.

Referring to FIG. 7B, this diagram illustrates, in a front view, the cart 700, as shown in FIG. 7A, in accordance with an embodiment of the present disclosure. The cart 700 may further comprises a removable cabinet 704 resting on the top side of the base 504. The cabinet 704 may house a computing device. In one example, a portable computing device such as a laptop computer may rest on top of the cabinet 704. In another example, the cabinet 704 may have a portable computing device or a desktop computing device inside the cabinet 704, with a monitor, keyboard, mouse or other I/O devices accessible either on top of the cabinet 704 or on a front side of the cabinet 704. The cart 700 may further include an integrated memory storage device for connection to a computing the device. In one example, the integrated memory storage device may include an optical drive, an external hard disk drive, a CD drive, a DVD drive, or a Bluray drive, or any combination thereof. The integrated memory storage device may be built into the cabinet 704 and may be connected to the computing device using any suitable interface, such as a USB interface cable.

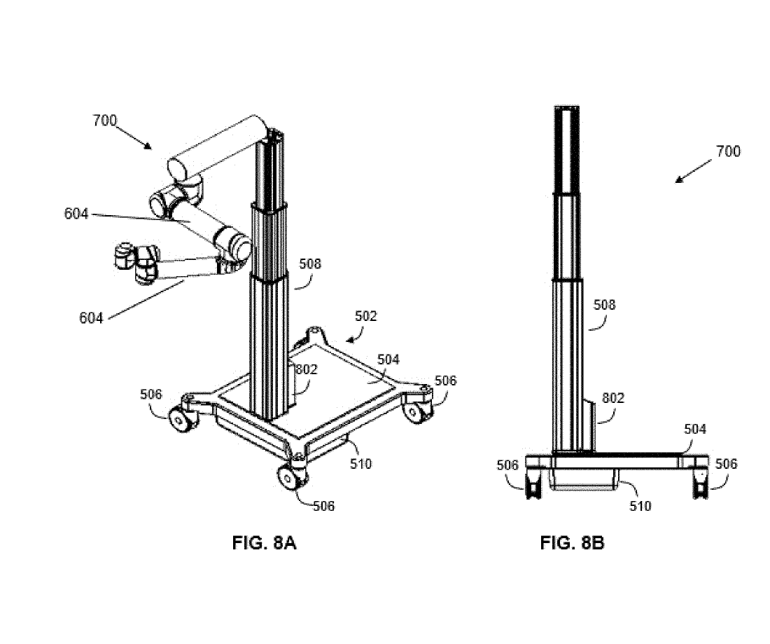

Referring to FIG. 8A, this diagram illustrates, in a perspective view, a cart 700 for housing components of a medical navigation system 205, in accordance with an alternative embodiment of the present disclosure. In this example, the cart 700 is shown with removable cabinet 704 having been removed. The cart 700 includes the frame 502, the base 504, the wheels 506, the column 508, the ballast 510, and the robotic arm 702. In one example, the column 508 is telescoping by way of electromechanical actuators (not shown) for lowering and raising the robotic arm 702. The electromechanical actuators may be connected to a control system that can be connected to a computing device using a suitable interface, such as a USB cable. In another example, the column 508 is modular and is at least partially hollow comprised of more than one columnar portion, such as three portions. The at least partially hollow column functions as a wire conduit for computing devices located in or on the cart 700.

Referring to FIG. 8B, this diagram illustrates, in a side view, the cart 700, as shown in FIG. 8A, in accordance with an embodiment of the present disclosure. The cart 700 further comprises an uninterrupted power supply or UPS 802. The UPS 802 may be located on the top side of the base 504 adjacent the column 508 and the UPS 802 may be coupled to the computing device 512 for supplying uninterrupted power to the computing device 512. Since the UPS is typically heavy, it may also aid the ballast 510 to stabilize the cart 700. While the example shown in FIG. 8 shows the ballast 510 on the bottom side of the base 504 and the UPS 802 on the top side of the base 504, both the UPS 802 and the ballast 510 may be located in either location either separately or together, depending on the design criteria of a particular application.

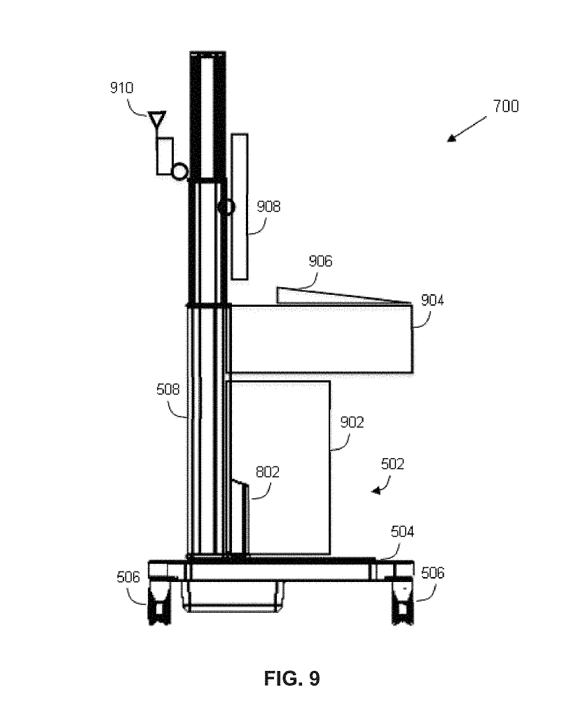

Referring to FIG. 9, this diagram illustrates, in a side view, the cart 700, as shown in FIG. 8A, with additional components, in accordance with an embodiment of the present disclosure. The cart 700 includes the frame 502, the base 504, the wheels 506, the column 508, the ballast 510, and the UPS 802. The cart 700 may further include a storage cabinet 902 resting on top of the top side of the base 504. In one example, the storage cabinet 902 may be removably attached to the cart 700. The cart 700 may further include a computer 904 attached to the column 508. The computer 904 may be either directly attachable to the column 508 or may be housed in a cabinet attached to the column 508. The computer 904 may have a keyboard 906 or input devices resting on top of the computer 904. The cart 700 may further have a display 908 attached to the column 508 and a wireless communications module 910 attached to the column 508. The UPS 802, computer 904, keyboard 906, display 908, and wireless communications module 910 may all be interconnected or coupled by wires that may use the conduit inside the column 508, as appropriate, for hiding the wires.

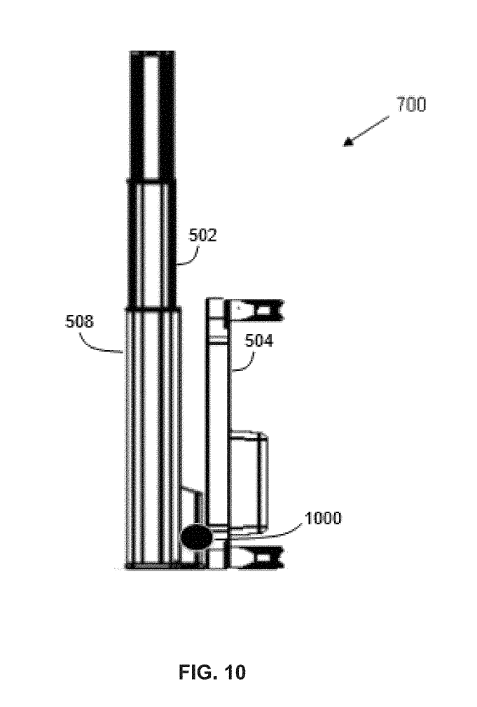

Referring to FIG. 10, this diagram illustrates, in a side view, a modification to the cart 700, as shown in FIG. 8A, in accordance with an embodiment of the present disclosure. In this example, the frame 502 further includes a hinge 1000 attaching the column 508 to the base 504 such that the column 508 is foldable relative to the base 504. While the hinge 1000 has been used as an example of a mechanism to facilitate folding, any suitable mechanism may be used to meet the criteria of a particular application and is encompassed by the present disclosure.

Still referring to FIG. 10 and back to FIGS. 5 and 6, in yet another example, the carts 500 and 600 may be provided together as a single combined cart, e.g., a cart assembly. A medical navigation system 205 may include a first cart 500 for housing components of the medical navigation system. The first cart 500 comprises a frame including a substantially horizontal base 504 having a bottom side and a top side with wheels 506 attached to the bottom side, a substantially vertical column 508 attached to the top side of the base 504, and a ballast 510 attached to the base 504 to function as a counterweight to avoid tipping of the cart 500. The components of the medical navigation system include a computing device 512 attached to the frame 502, the computing device 512 having a processor coupled to a memory and a wireless communication component for communicating wirelessly with a computing device associated with a second cart 600. The second cart 600 may be for housing further components of the medical navigation system. The second cart 600 comprises a second frame 502 including a second substantially horizontal base 504 having a bottom side and a top side with wheels 506 attached to the bottom side, a second substantially vertical column 508 attached to the top side of the base, a second ballast 510 attached to the base 504 to function as a counterweight to avoid tipping of the cart 600, and a display 602 of at least 55 inches in diagonal size mounted on the second substantially vertical column 508.

Still referring to FIG. 10 and back to FIGS. 5 and 6, in one example, the cart 500 may be referred to as a navigation cart and the auxiliary cart 600 may be referred to as an auxiliary cart. The two separate carts 500 and 600 allow the surgeon to position the cart 600 with the tracking camera 610 and monitor 602 in the optimal position with respect to his patient and site of operation. The navigation cart 500 may hold the computing device 512 and may have an operator and may or may not need to be close to the auxiliary cart 600. Therefore, the cart 500 may be positioned as far as 30 feet away from the auxiliary cart 600 and away from other instruments to reduce clutter in an operating room. The computing device 512 may be laptop computer that is easy to upgrade. The computing device 512 may sit on top of the shelf 514 and switching computing devices 512 may be a simple as switching cables. The carts 500, 600, and 700 may be configured where each main component is modular and may be easily removed and upgradeable.

Still referring to FIG. 10 and back to FIGS. 5 and 6, any of the carts 500, 600, and 700 may include a computing device such as a laptop, keyboard and mouse, an isolation transformer, an NDI USB Hub for Polaris Spectra, power adaptors for the laptop and Polaris camera, an optical drive and USB port, a Polaris Spectra tracking camera, an NDS 55″ G2 Radiance medical grade monitor, Polaris Spectra adaptor and handle to the arm, and/or a storage drawer. Enclosures such as the enclosure 704 may be designed to accommodate one or more of an isolation transformer, an NDI USB hub, a DVD, Blu-Ray or other Optical drive, a USB drive, a patient reference adaptor and extension arm to Mayfield clamp, provide for cable strain relief, and/or port panel, and/or a power adaptor enclosure. Computing device may also include a desktop personal computer (PC), a tablet, Smartphone and/or an embedded computing device.

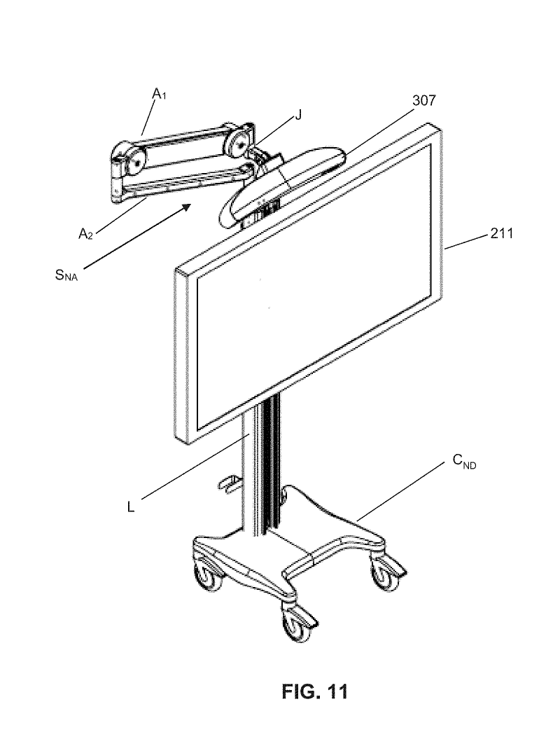

Referring to FIG. 11, this diagram illustrates, in a perspective view, a navigation arm system S_{NA}, as undeployed or stored, operable with a navigation display cart C_{ND} and a navigation display device, such as a camera 307, of a navigation system, such as the medical navigation system 205, in accordance with an embodiment of the present disclosure. The navigation arm system S_{NA} for supporting the navigation display device, such as the camera 307, of the navigation system, such as the medical navigation system 205, is capable of coupling with a navigation display cart C_{ND}. The navigation arm system S_{NA} comprises: an arm assembly A_{A} configured to position the navigation device, the arm assembly A_{A} comprising: a first arm linkage A₁ having a tension adjustment feature (not shown) and releasably connectable to the navigation device; a second arm linkage A₂ capable of connecting the arm assembly A_{A} to the navigation display cart C_{ND}; and at least one joint J coupling at least the first and second arm linkages A₁, A₂; and a locking mechanism L configured to maintain a disposition of the arm assembly A_{A} in relation to the navigation display cart C_{ND} in a locked, stored, position and an unlocked, deployed, position, the locking mechanism L configured to lockably couple the arm assembly A_{A} in relation to the navigation display cart C_{ND}, the locking mechanism L comprising a plurality of locking modes, whereby structural stability is providable. By example only, the navigation device comprises a camera 307, such as an optical camera.

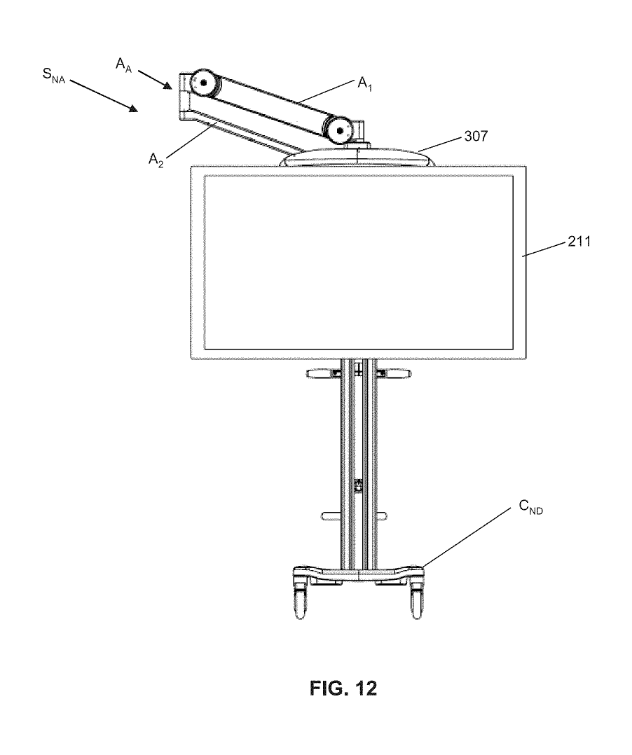

Referring to FIG. 12, this diagram illustrates, in a front view, a navigation arm system S_{NA}, as undeployed, or stored, operable with a navigation display cart C_{ND} and a navigation display device, such as the camera 307, of navigation system, such as the medical navigation system 205, in accordance with an embodiment of the present disclosure. In the undeployed, stored, position, the arm assembly A_{A} of the navigation arm system S_{NA} is neatly “tucked” behind the display device 211. A longitudinal axis of the second arm linkage A₂ subtends a horizontal line corresponding to an upper edge of the display device 211 at an angle Θ of approximately 20 degrees, whereby the arm assembly A_{A} is configured to cleat the display device 211 when the navigation arm system S_{NA} is deployed for operation. In this embodiment, the angle is preferable at 20 degrees, however other angles in the range of 10 degrees to 30 degrees may be considered.

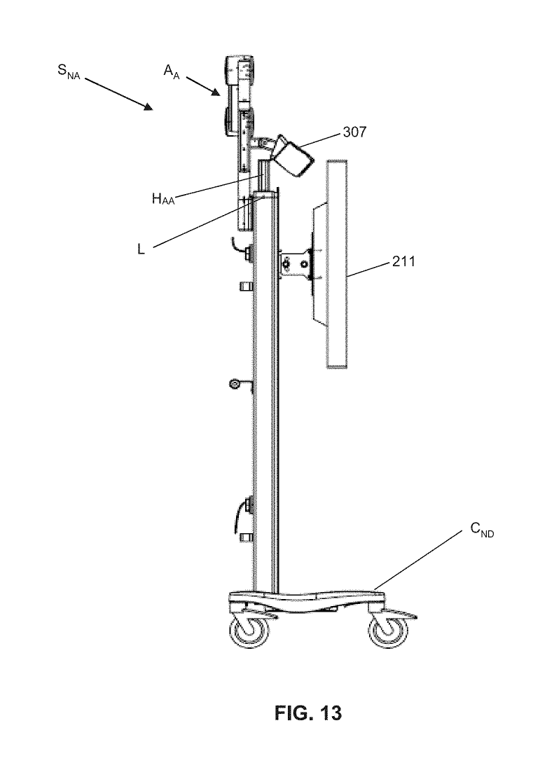

Referring to FIG. 13, this diagram illustrates, in a side view, a navigation arm system S_{NA}, as undeployed or stored, operable with a navigation display cart C_{ND} and a navigation display device, such as the camera 307, of navigation system, such as the medical navigation system 205, in accordance with an embodiment of the present disclosure. In the undeployed, stored, position, the arm assembly A_{A} of the navigation arm system S_{NA} is neatly “tucked” behind the plane of the display device 211, as shown in this side view, wherein collisions in the medical environment, such as an operating room, are minimized during transportation of the navigation display cart C_{ND}.

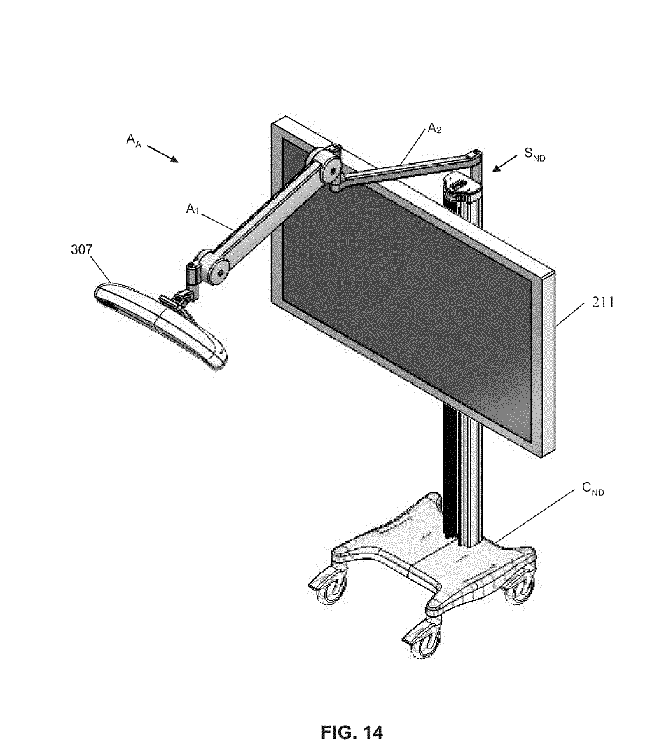

Referring to FIG. 14, this diagram illustrates, in a perspective view, a navigation arm system S_{NA}, as deployed, operable with a navigation display cart C_{ND} and a navigation display device, such as the camera 307, of navigation system, such as the medical navigation system 205, in accordance with an embodiment of the present disclosure. The A_{A} of the navigation arm system S_{NA} is deployable in operation and retractable during non-operation.

Referring to FIG. 15, this diagram illustrates, in a front view of a navigation arm system S_{NA}, as deployed, operable with a navigation display cart C_{ND} and a navigation display device, such as the camera 307, of navigation system, such as the medical navigation system 205, in accordance with an embodiment of the present disclosure. The arm assembly A_{A} of the navigation arm system S_{NA} is deployable in operation and retractable during non-operation.

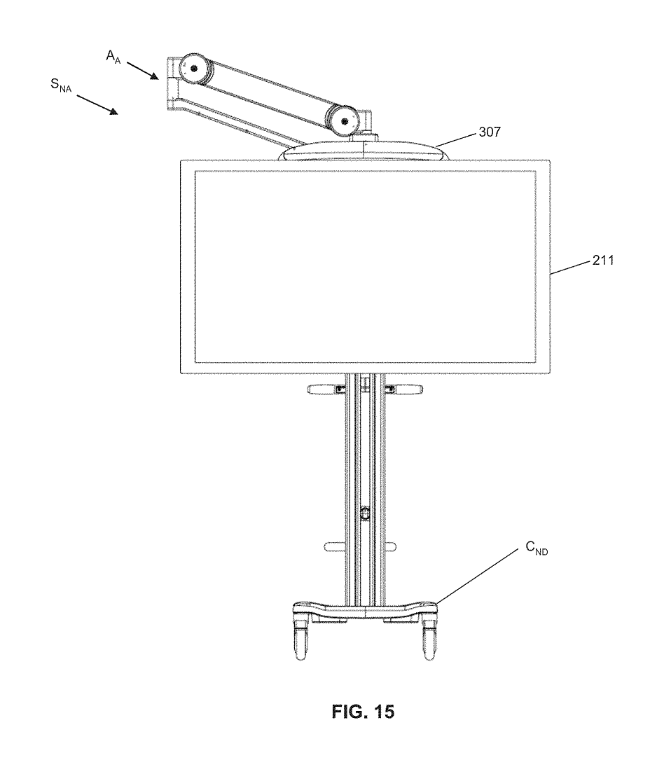

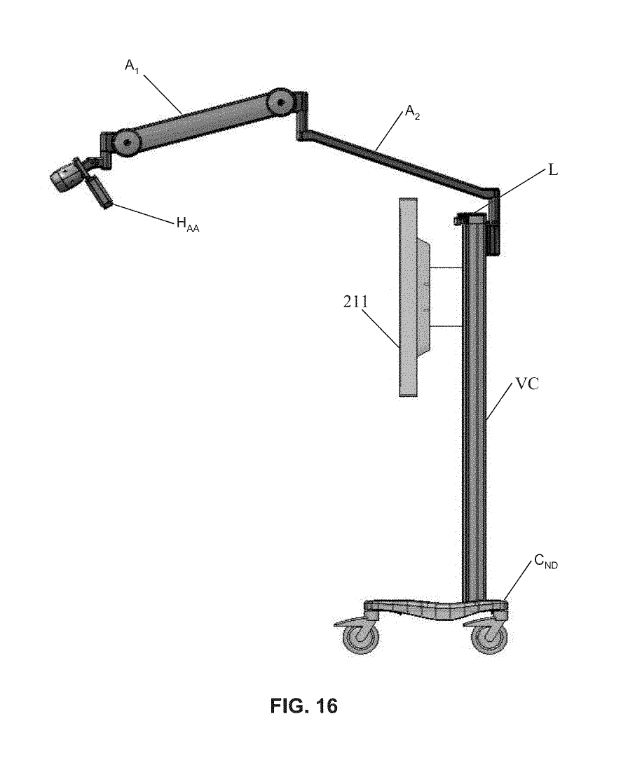

Referring to FIG. 16, this diagram illustrates, in a side view, a navigation arm system S_{NA}, as deployed, operable with a navigation display cart C_{ND} and a navigation display device, such as the camera 307, of navigation system, such as the medical navigation system 205, in accordance with an embodiment of the present disclosure. The locking mechanism L is configured to couple with a vertical column VC or spine of the navigation display cart C_{ND}.

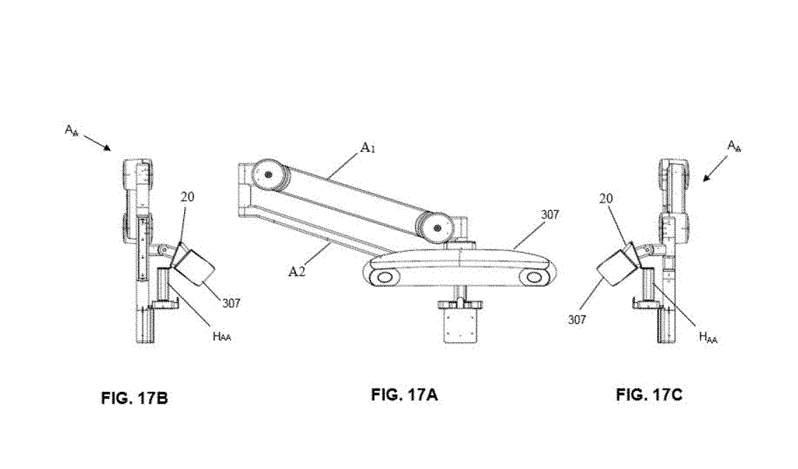

Referring to FIG. 17A, this diagram illustrates, in a front view, a navigation arm system S_{NA}, in the undeployed or stored disposition, as shown FIG. 11, in accordance with an embodiment of the present disclosure. The second arm linkage A₂ is configured for disposition in an obtuse angle (in relation to a longitudinal axis of the vertical column VC), whereby, when in a deployed position, the arm assembly A_{A} is capable of clearing the navigation display device 211, such as the camera 307, when disposed in relation to the navigation display cart C_{ND}. The obtuse angle Φ comprises a range of at least approximately 120 degrees.

Referring to FIG. 17B, this diagram illustrates, in a side view, a navigation arm system S_{NA}, in the undeployed or stored disposition, as shown FIG. 11, in accordance with an embodiment of the present disclosure. The arm assembly A_{A} further comprises an arm assembly handle H_{AA} for facilitating manual operation of the arm assembly A_{A}. The arm assembly handle H_{AA} is configured to engage with the locking mechanism L when the navigation arm system S_{NA} is undeployed.

Referring to FIG. 17C, this diagram illustrates, in an opposing side view, a navigation arm system S_{NA}, in the undeployed or stored disposition, as shown FIG. 11, in accordance with an embodiment of the present disclosure. The arm assembly A_{A} further comprises an arm assembly handle H_{AA} for facilitating manual operation of the arm assembly A_{A}. The arm assembly handle H_{AA} is configured to engage with the locking mechanism L when the navigation arm system S_{NA} is undeployed.

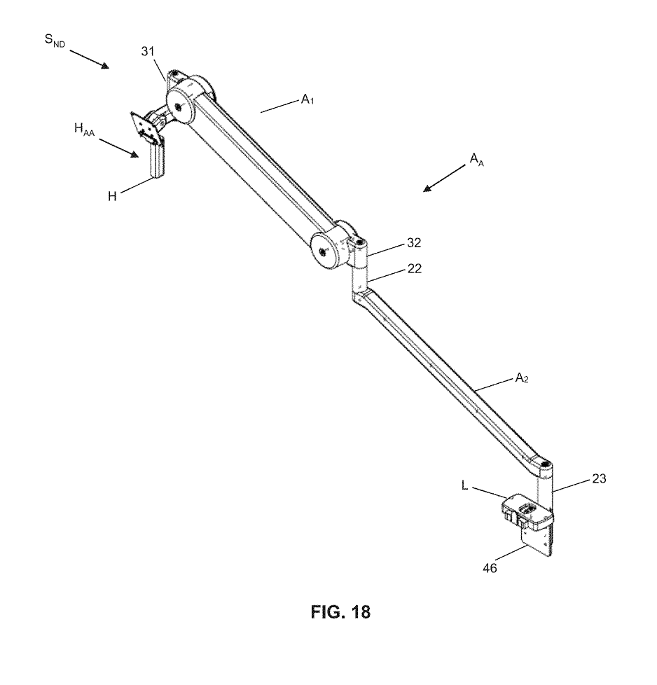

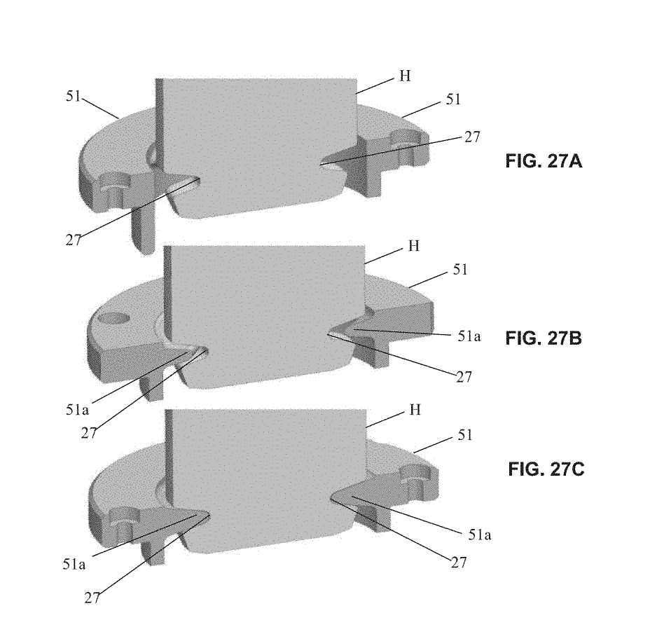

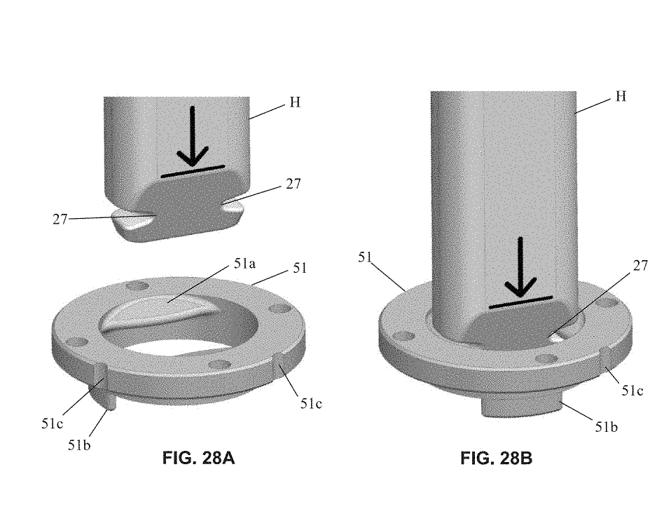

Referring to FIG. 18, this diagram illustrates, in a perspective view, a navigation arm system S_{NA}, in the deployed disposition, as shown FIG. 14, in accordance with an embodiment of the present disclosure. The arm assembly A_{A} further comprises an arm assembly handle H_{AA} for facilitating manual operation of the arm assembly A_{A}. The arm assembly handle H_{AA} is configured to engage with the locking mechanism L when the navigation arm system S_{NA} is undeployed. The arm assembly handle H_{AA} is further configured to couple with the navigation device mounting bracket 20, the arm assembly handle H_{AA} comprising at least one notch 27 (FIGS. 27A-28B) and configured to lockably couple with the locking mechanism L and to facilitate positioning of the navigation device, such as the camera 307.

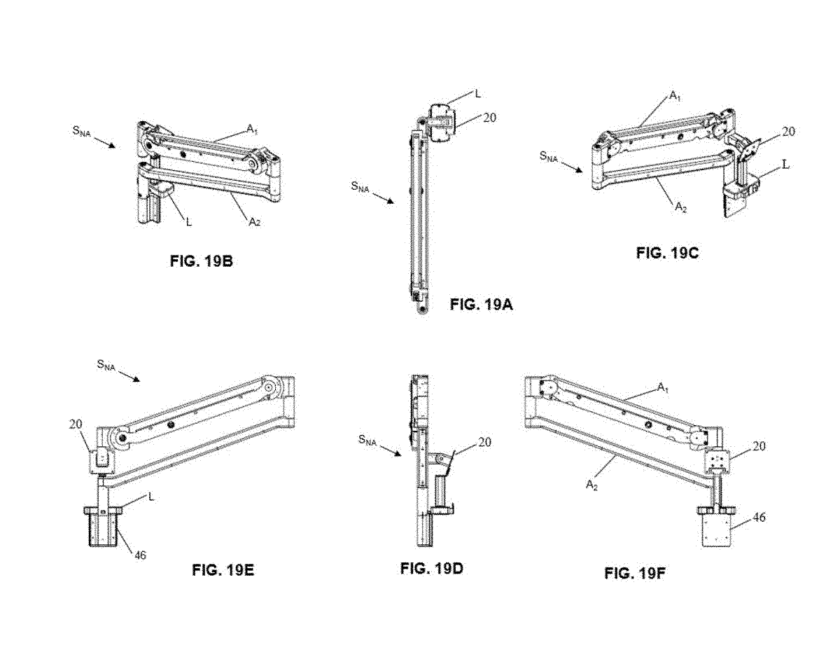

Referring to FIGS. 19A-19F, with respect to FIG. 19A, this diagram illustrates, in a top view, a navigation arm system S_{NA}, in the undeployed or stored disposition, as shown FIG. 11, in accordance with an embodiment of the present disclosure. Referring to FIG. 19B, this diagram illustrates, in a rear perspective view, a navigation arm system S_{NA}, in the undeployed or stored disposition, as shown FIG. 11, in accordance with an embodiment of the present disclosure. Referring to FIG. 19C, this diagram illustrates, in a rear perspective view, a navigation arm system S_{NA}, in the undeployed or stored disposition, as shown FIG. 11, in accordance with an embodiment of the present disclosure. Referring to FIG. 19D, this diagram illustrates, in a side view, a navigation arm system S_{NA}, in the undeployed or stored disposition, as shown FIG. 11, in accordance with an embodiment of the present disclosure. Referring to FIG. 19D, this diagram illustrates, in a rear view, a navigation arm system, in the undeployed or stored disposition, as shown FIG. 11, in accordance with an embodiment of the present disclosure. Referring to FIG. 19E, this diagram illustrates, in a rear view, a navigation arm system S_{NA}, in the undeployed or stored disposition, as shown FIG. 11, in accordance with an embodiment of the present disclosure. Referring to FIG. 19F, this diagram illustrates, in a front view, a navigation arm system S_{NA}, in the undeployed or stored disposition, as shown FIG. 11, in accordance with an embodiment of the present disclosure.

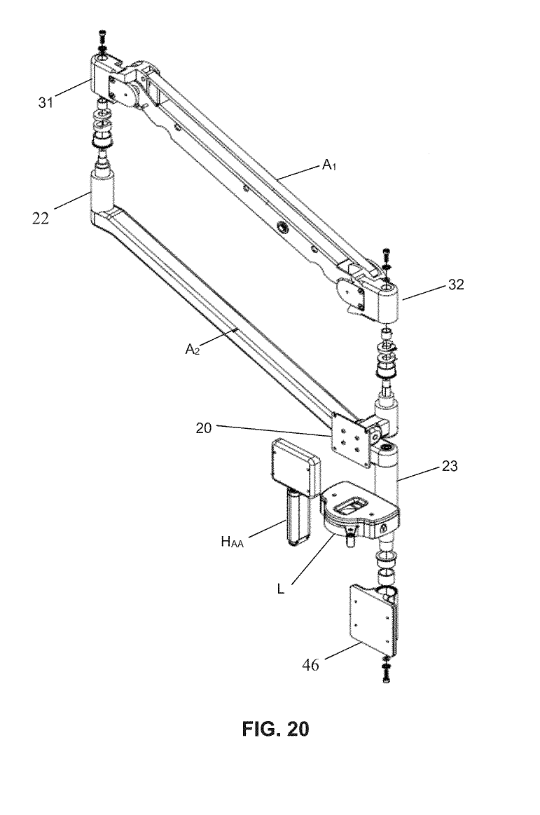

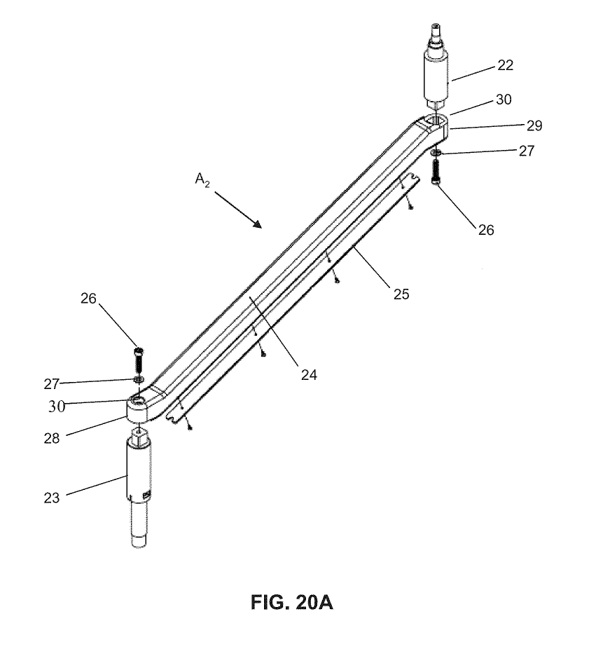

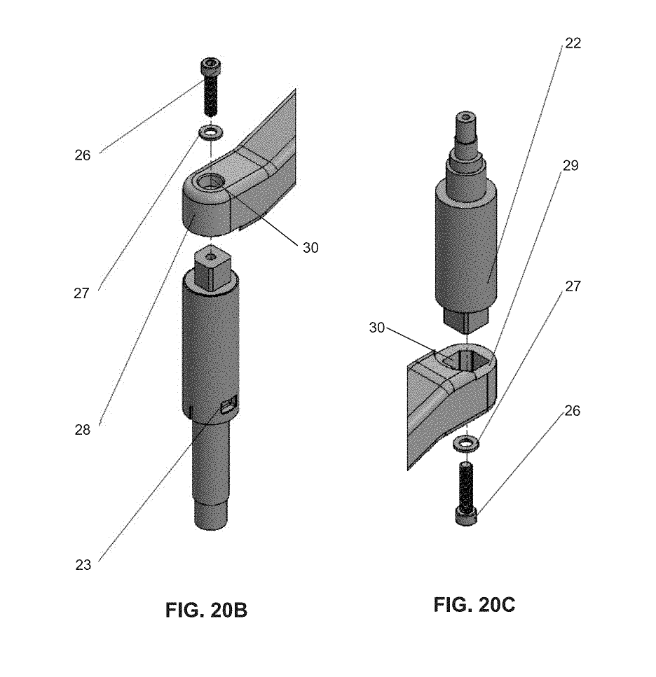

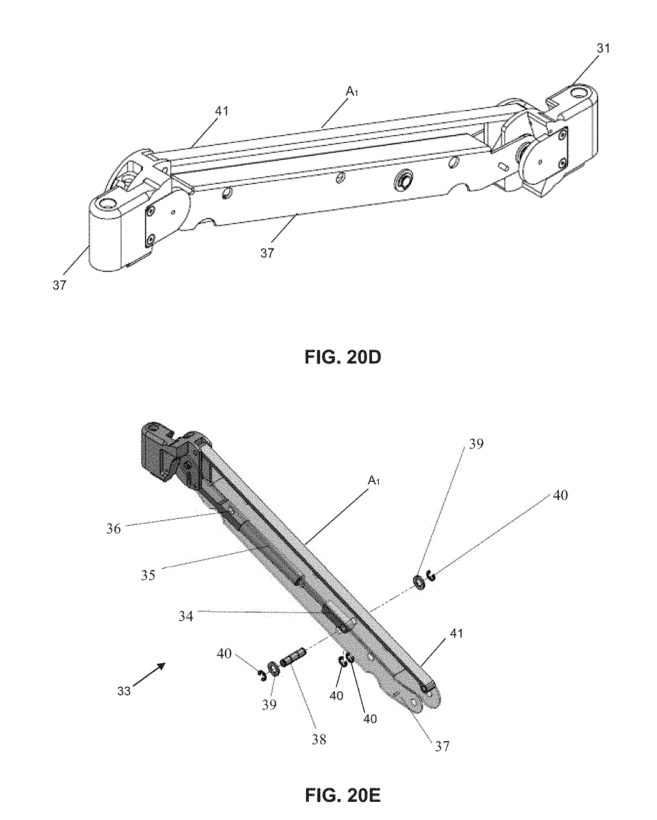

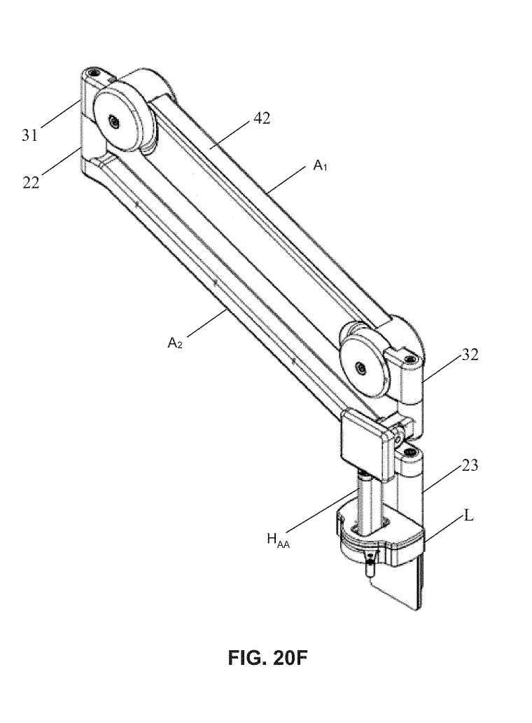

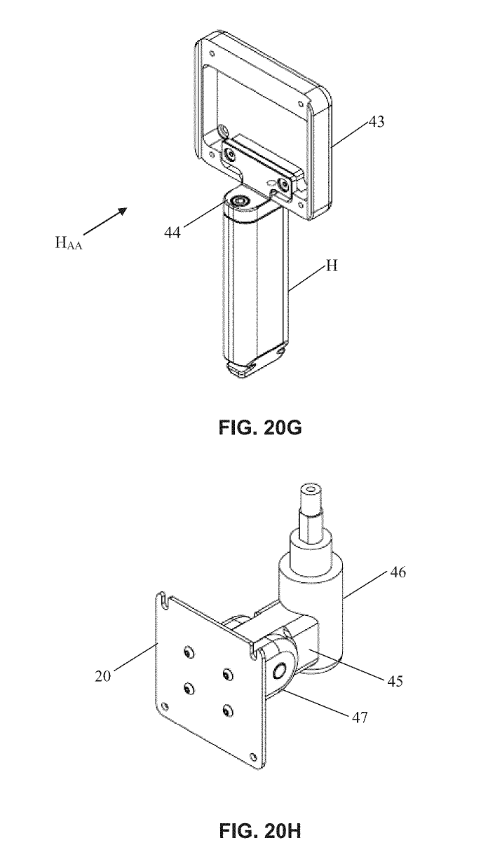

Referring to FIG. 20, this diagram illustrates, in an exploded perspective view, a navigation arm system S_{NA}, in the undeployed or stored disposition, as shown FIG. 11, in accordance with an embodiment of the present disclosure. The system S_{NA} further comprises a plurality of mounting brackets, the plurality of mounting brackets comprising the navigation device mounting bracket 20, configured to accept the navigation device, such as the camera 307, and a cart mounting bracket 21 configured to couple the arm assembly A_{A} in relation to the navigation display cart C_{ND}.

Referring to FIG. 20A, this diagram illustrates, in an exploded perspective view, a second arm linkage A₂, or rigid arm, of an arm assembly A_{A} in a navigation arm system S_{NA}, as shown FIG. 20, in accordance with an embodiment of the present disclosure. In the system S_{NA}, the second arm linkage A₂ is configured to couple the arm assembly A_{A} with a top portion of the vertical column VC or spine of the navigation display cart C_{ND}. The second arm linkage A₂ comprises a rigid extension arm 24 and a cable retention arm 25. The navigation arm system S_{NA} further comprises at least one stem for facilitating joining the second arm linkage A₂ with at last one of the first arm linkage Aₗ and the navigation device, such as the camera 307. The at least one stem comprises at least one of an arm joint stem 22 and an extension arm stem 23, and at least one fastener, such as a threaded fastener 26, a washer 27, and the like.

Referring to FIG. 20B, this diagram illustrates, in an exploded perspective view, an extension arm stem 23 in a navigation arm system S_{NA}, as shown FIG. 20, in accordance with an embodiment of the present disclosure. The rigid extension arm 24 comprises a proximal end 28 and a distal end 29, each of which comprises a bore 30 through which at least one fastener, such as a threaded fastener 26, a washer 27, e.g., a locking washer, and the like, are used to fasten the at least one stem thereto. The extension arm stem 23 is configured to couple with the cart mounting bracket 21.

Referring to FIG. 20C, this diagram illustrates, in an exploded perspective view, an arm joint stem 22 of a navigation arm system S_{NA}, as shown FIG. 20, in accordance with an embodiment of the present disclosure. The rigid extension arm 24 comprises a proximal end 28 and a distal end 29, each of which comprises a bore 30 through which at least one fastener, such as a threaded fastener 26, a washer 27, e.g., a locking washer, and the like, are used to fasten the at least one stem thereto. The arm joint stem 22 is configured to couple with a first arm linkage joint 31.

Referring to FIG. 20D, this diagram illustrates, in a perspective view, a first arm linkage A₁, or a spring arm, of an arm assembly A_{A} in a navigation arm system S_{NA}, as shown FIG. 20, in accordance with an embodiment of the present disclosure. The navigation arm system S_{NA} further comprises at least one joint, such as a first arm linkage joint 31 and a navigation device mounting joint 32. The joints are configured to rotate about the stems, in accordance with embodiments of the present disclosure, wherein the joints may be lubricated to facilitate such rotation about the stems.

Referring to FIG. 20E, this diagram illustrates, in a exploded cut-away perspective view, a first arm linkage A₁, or a spring arm, of an arm assembly A_{A} in a navigation arm system S_{NA}, as shown FIG. 20, in accordance with an embodiment of the present disclosure. The first arm linkage Aₗ comprises a fixed bottom linkage 37 and a fixed linkage top 41, a tension adjustment feature 33 disposed within the fixed bottom linkage 37, wherein the tension adjustment feature 33 comprises a piston, such as a hydraulic piston 34, and a gas-driven spring 35, whereby the first arm linkage A₁ is configured to counterweight the navigation device, such as the camera 307, and whereby navigation integrity is maintainable. The gas-driven spring 35 is coupled with the fixed bottom linkage 37 by way a pin or dowel 38, washers 39, and clips 40, by example only. The tension adjustment feature 33 is further adjustable and can accommodate a variety of navigation devices having a variety of weights.

Referring to FIG. 20F, this diagram illustrates, in a exploded perspective view, a first arm linkage A₁, or a spring arm, of an arm assembly A_{A} in a navigation arm system S_{NA}, as shown FIG. 20, in accordance with an embodiment of the present disclosure. The first arm linkage A₁ comprises a fixed bottom linkage 37 and a fixed linkage top 41, a tension adjustment feature 33 disposed within the fixed bottom linkage 37, wherein the tension adjustment feature 33 comprises a piston, such as a hydraulic piston 34, and a gas-driven spring 35, whereby the first arm linkage A₁ is configured to counterweight the navigation device, such as the camera 307, and whereby navigation integrity is maintainable. The gas-driven spring 35 is coupled with the fixed bottom linkage 37 by way a pin or dowel 38, washers 39, and clips 40, by example only. The tension adjustment feature 33 is further adjustable and can accommodate a variety of navigation devices having a variety of weights.

Still referring to FIG. 20F, the first arm linkage A₁, or a spring arm, further comprises a cover member 42, of an arm assembly A_{A} in a navigation arm system S_{NA}, as shown FIG. 20, in an undeployed position. The cover member 42 protects internal moving components of the first arm linkage A₁ against external elements, such as dust, dirt, fluids, and the like. The cover member 42 also comprises a cable management feature for routing and securing a camera cable through a designated route to a destination. Referring to FIG. 20H, this diagram illustrates, in a perspective view, a first arm linkage A₁, or a spring arm, further comprising a cover member 42, of an arm assembly A_{A} in a navigation arm system S_{NA}, as shown FIG. 20, in an undeployed position, in accordance with an embodiment of the present disclosure. A navigation device mounting joint 32 or a main joint of an arm assembly A_{A} in a navigation arm system S_{NA} is also shown. The navigation device mounting joint 32 is coupled with the first arm linkage A₁ by way of a variety of fasteners, as shown by example only. The first arm linkage joint 31 is coupled with the first arm linkage A₁ by way of a variety of fasteners, as shown by example only.

Referring to FIG. 20G, this diagram illustrates, in a perspective view, an assembly handle H_{AA} of an arm assembly A_{A} in a navigation arm system S_{NA}, as shown FIG. 20, in accordance with an embodiment of the present disclosure. The assembly handle H_{AA} comprises a handle portion H, an adapter 43 for accommodating a variety of navigation devices, such as an optical camera and a Raman spectroscopic device, and an adapter bracket 44 for facilitating coupling the adapter 43 with the handle portion H, e.g., by way of a variety of fasteners, as shown by example only. Referring to FIG. 20L, this diagram illustrates, in a perspective view, an assembly handle H_{AA} of an arm assembly A_{A} in a navigation arm system S_{NA}, as shown FIG. 20, in an assembled state, in accordance with an embodiment of the present disclosure.

Referring to FIG. 20H, this diagram illustrates, in a perspective view, a navigation device mounting joint 45 of an arm assembly A_{A} in a navigation arm system S_{NA}, as shown FIG. 20, in accordance with an embodiment of the present disclosure. The navigation device mounting joint 45 facilitates coupling of the mounting plate 20 with a navigation device stem 46 by way of a variety of fasteners, as shown by example only. The mounting plate 20 is configured to accept the adapter 43. A pivot arm 47 is pivotally coupled with the navigation device mounting joint 45. The mounting plate 20 is coupled with the pivot arm 47.

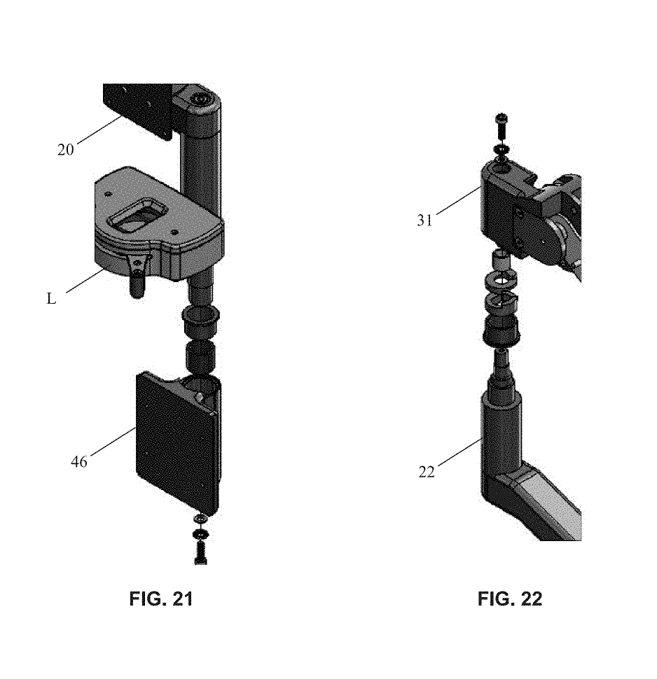

Referring to FIG. 21, this diagram illustrates, in an exploded perspective view, a mounting bracket 46 and a locking mechanism L of a navigation arm system S_{NA}, in accordance with an embodiment of the present disclosure. The locking mechanism L is further configured to lockably couple the arm assembly handle H_{AA} in relation to the navigation display cart C_{ND}, whereby a profile of the arm assembly A_{A} is minimized during transportation of the navigation display cart C_{ND}. The locking mechanism L is further configured to maintain a disposition of the navigation device, such as the camera 307, in relation to the arm assembly A_{A}.

Referring to FIG. 22, this diagram illustrates, in an exploded perspective view, a first arm linkage joint 31 for rotatably coupling first and second arm linkages A₁, A₂ of an arm assembly A_{A} in a navigation arm system S_{NA}, in accordance with an embodiment of the present disclosure. The first arm linkage joint 31 is rotatable about the stem 22.

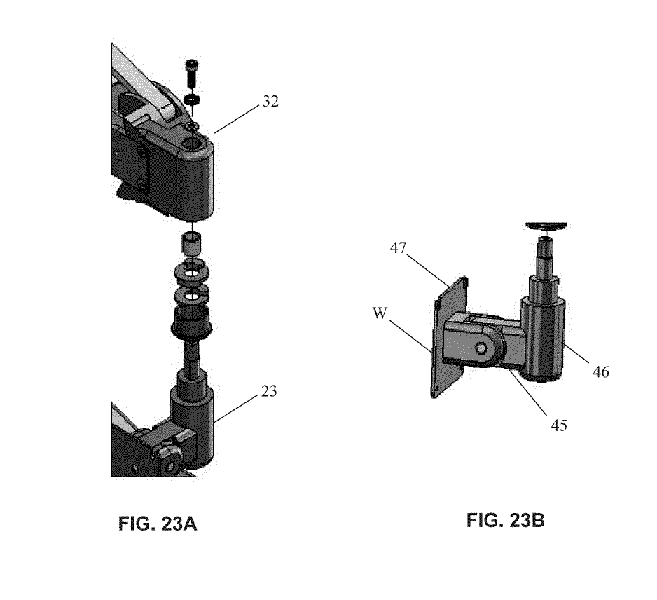

Referring to FIG. 23A, this diagram illustrates, in an exploded perspective view, navigation device mounting joint 32 for rotatably coupling a first arm linkage A₁, of an arm assembly A_{A} in a navigation arm system S_{NA}, with a navigation device, such as the camera 307, in accordance with an embodiment of the present disclosure. The navigation device mounting joint 32 is rotatable about the stem 23.

Referring to FIG. 23B, this diagram illustrates, in a perspective view, a navigation device mounting joint 45 for rotatably coupling a first arm linkage A₁, of an arm assembly A_{A} in a navigation arm system S_{NA}, with a navigation device, such as the camera 307, in accordance with an embodiment of the present disclosure. The navigation device mounting joint 45 facilitates coupling of the mounting plate 20 with a navigation device stem 46 by way of a variety of fasteners, as shown by example only. The mounting plate 20 is configured to accept the adapter 43. A pivot arm 47 is pivotally coupled with the navigation device mounting joint 45. The mounting plate 20 is coupled with the pivot arm 47.

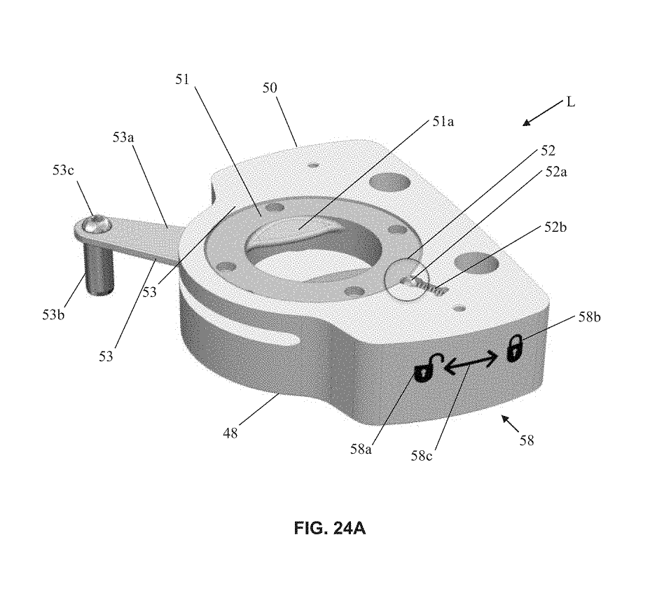

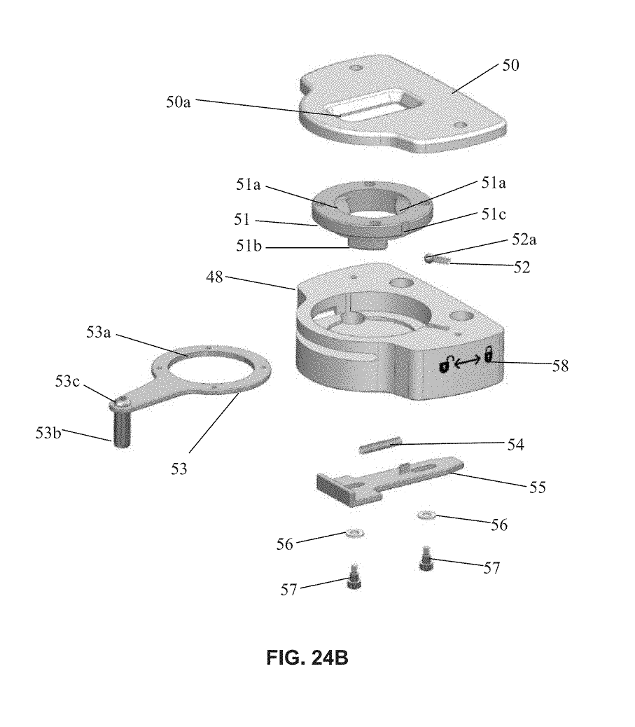

Referring to FIG. 24A, this diagram illustrates, in a perspective view, a locking mechanism L in a navigation arm system S_{NA}, in accordance with an embodiment of the present disclosure. The locking mechanism comprises a base 48 having a slit 49, a cap 50, a lock ring 51, at least one ball-and-compression-spring assembly 52, a ring handle 53, a compression spring 54, a lock tab 55, at least one washer 56, and at least one bolt 57, e.g., at least one shoulder bolt, the base 48 accommodating the cap 50, the lock ring 51, the at least one ball-and-compression-spring assembly 52, the ring handle 53, the compression spring 54, the lock tab 55, the at least one washer 56, and the at least one bolt 57 (FIGS. 24B and 24C). The base 48 further comprises indicia 58 for indicating a locked position and an unlocked position to guide a user in operating the ring handle 53. The indicia 58 comprise an icon 58a, representing an unlocked padlock, and an icon 58b, representing a locked padlock, and a double-headed arrow 58c, representing respective directions for locking and unlocking the locking mechanism L. The lock ring 51 comprises at least one cam portion 51a that is engageable with the at least one notch of a handle portion H of the arm assembly handle H_{AA}. The ring handle 53 comprises a ring portion 53a and a handle portion 53b which are either integrally formed or coupled together using at least one fastener, such as bolt 53c. During operation, a ball detent 52a engages notches 51c of the lock ring 51.

Referring to FIG. 24B, this diagram illustrates, in an exploded perspective view, a locking mechanism L in a navigation arm system S_{NA}, in accordance with an embodiment of the present disclosure. The locking mechanism comprises a base 48 having a slit 49, a cap 50 having a slot 50a, a lock ring 51, at least one ball-and-compression-spring assembly 52, a ring handle 53, a compression spring 54, a lock tab 55, at least one washer 56, and at least one bolt 57, e.g., at least one shoulder bolt, the base 48 accommodating the cap 50, the lock ring 51, the at least one ball-and-compression-spring assembly 52, the ring handle 53, the compression spring 54, the lock tab 55, the at least one washer 56, and the at least one bolt 57 (FIGS. 24B and 24C). The base 48 further comprises indicia 58 for indicating a locked position and an unlocked position to guide a user in operating the ring handle 53. The indicia 58 comprise an icon 58a, representing an unlocked padlock, and an icon 58b, representing a locked padlock, and a double-headed arrow 58c, representing respective directions for locking and unlocking the locking mechanism L. The lock ring 51 comprises at least one cam portion 51a that is engageable with the at least one notch of a handle portion H of the arm assembly handle H_{AA}. The ring handle 53 comprises a ring portion 53a and a handle portion 53b which are either integrally formed or coupled together using at least one fastener, such as bolt 53c.

Still referring to FIG. 24B, the ring handle 53 comprises: a ring portion 53a operably engageable with the lock ring 51 and the lock tab 55; and a ring handle portion 53b coupled with the ring portion 53a and disposable through the slit 49 of the base 48, the ring handle 53 configured to contemporaneously engage and contemporaneously disengage at least one flange 51b of the ring lock 51 in relation to at least one notch 27 of handle portion H of the arm assembly handle H_{AA} by way of the at least one ball-and-compression-spring assembly 52 and the lock tab 55 in relation to a vertical column VC or a spine of the navigation display cart C_{ND} by way of the compression spring 54 and a cam 51a. The compression spring 54 is configured to urge the lock tab 55 outboard in relation to the base 48, and wherein the cam 51a engages at least one notch 27 and the flange 51b urges the lock tab 55 inboard against the compression spring 54 and in relation to the base 48.

Referring to FIG. 25A, this diagram illustrates, in a top view, a ring handle 53 in a locking mechanism L, disposed in a unlocked position, wherein the lock tab 55 is undeployed, in accordance with an embodiment of the present disclosure. The cap 50 is removed to facilitate understanding of the underlying components and their functions. Actuating the ring handle 53 toward the “unlocking” position disengages the at least one “cam” or flange 51b of the lock ring 51, thereby compressing the compression spring 54, and thereby pulling back or undeploying the lock tab 55.

Referring to FIG. 25B, this diagram illustrates, in a top view, a ring handle 53 in a locking mechanism L, disposed in an locked position, wherein the lock tab 55 is deployed, in accordance with an embodiment of the present disclosure. The cap 50 is removed to facilitate understanding of the underlying components and their functions. Actuating the ring handle 53 toward the “locking” position engages the at least one “cam” or flange 51b of the lock ring 51, thereby releasing the compression spring 54, thereby urging or deploying the lock tab 55.

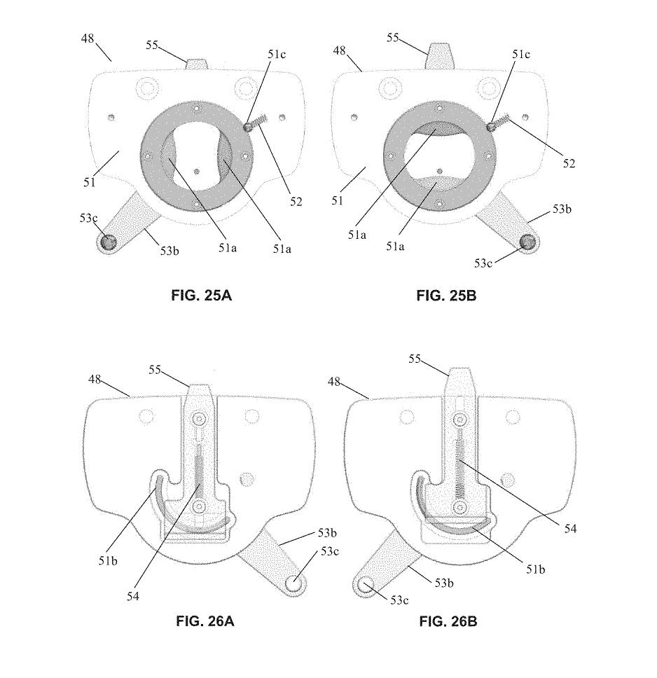

Referring to FIG. 26A, this diagram illustrates, in a bottom view, a ring handle 53 in a locking mechanism L, disposed in a unlocked position, wherein the lock tab 55 is undeployed, in accordance with an embodiment of the present disclosure. Actuating the ring handle 53 toward the “unlocking” position disengages the at least one “cam” or flange 51b of the lock ring 51, thereby compressing the compression spring 54, and thereby pulling back or undeploying the lock tab 55.

Referring to FIG. 26B, this diagram illustrates, in a bottom view, a ring handle 53 in a locking mechanism L, disposed in an locked position, wherein the lock tab 55 is deployed, in accordance with an embodiment of the present disclosure. Actuating the ring handle 53 toward the “locking” position engages the at least one “cam” or flange 51b of the lock ring 51, thereby releasing the compression spring 54, thereby urging or deploying the lock tab 55.

Referring to FIG. 27A, this diagram illustrates, in a cut-away perspective view, a ring lock 51 in a locking mechanism L interacting with a handle portion H of an arm assembly handle H_{AA}, disposed in a fully unlocked position, wherein the at least one cam 51a of the lock ring 51 is disengaged from the at least one notch 27 of the handle portion H, in accordance with an embodiment of the present disclosure.

Referring to FIG. 27B, this diagram illustrates, in a cut-away perspective view, a ring lock 51 in a locking mechanism L interacting with a handle portion H of an arm assembly handle H_{AA}, disposed in a partially locked position, wherein the at least one cam 51a of the lock ring 51 is partially engaged with the at least one notch 27 of the handle portion H, in accordance with an embodiment of the present disclosure.

Referring to FIG. 27C, this diagram illustrates, in a cut-away perspective view, a ring lock 51 in a locking mechanism L interacting with a handle portion H of an arm assembly handle H_{AA}, disposed in a fully locked position, wherein the at least one cam 51a of the lock ring 51 is fully engaged with the at least one notch 27 of the handle portion H, in accordance with an embodiment of the present disclosure.

Referring to FIG. 28A, this diagram illustrates, in a cut-away perspective view, a ring lock 51 in a locking mechanism L interacting with a handle portion H of an arm assembly handle H_{AA}, disposed in a fully unlocked position and ready to accept the handle portion H of an arm assembly handle H_{AA}, in accordance with an embodiment of the present disclosure. In the unlocked position, the handle H is not yet engaged while the lock tab 55 is not yet urged forward as the flange 51c is not yet pivoted into a position to release the compression spring 54.

Referring to FIG. 28B, this diagram illustrates, in a cut-away perspective view, a ring lock 51 in a locking mechanism L interacting with a handle portion H of an arm assembly handle H_{AA}, disposed in a fully locked position, having accepted the handle portion H of an arm assembly handle H_{AA}, in accordance with an embodiment of the present disclosure. In the locked position, the handle H is engaged while the lock tab 55 is urged forward as the flange 51c is pivoted into a position to release the compression spring 54. Accordingly, the locking mechanism L provides multi-modal locking, e.g., by way of both the cams 51a engaging the notches 27 as well as by way of simultaneously urging the lock tab 55 into a slot in the vertical column VC.

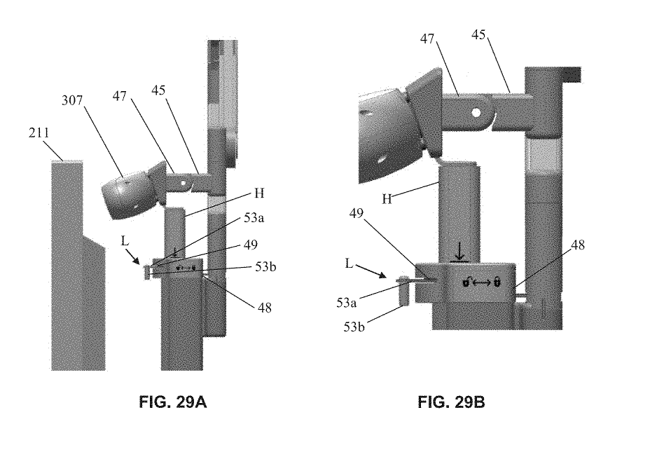

Referring to FIG. 29A, this diagram illustrates, in a side view, the locking mechanism L interacting with a handle portion H of an arm assembly handle H_{AA}, disposed in a fully locked position, having accepted the navigation device handle, in accordance with an embodiment of the present disclosure. The navigation device mounting joint 45 facilitates coupling of the mounting plate 20 with a navigation device stem 46 by way of a variety of fasteners, as shown by example only. The mounting plate 20 is configured to accept the adapter 43. A pivot arm 47 is pivotally coupled with the navigation device mounting joint 45. The mounting plate 20 is coupled with the pivot arm 47. The camera 307 is safely tucked behind the display device 211 when the navigation arm system is undeployed and locked in place by way of the locking mechanism L.

Referring to FIG. 29B, this diagram illustrates, in a close side view, the locking mechanism L interacting with a handle portion H of an arm assembly handle H_{AA}, disposed in a fully locked position, having accepted the navigation device handle, wherein the navigation device mounting joint is configured to make a hard stop for an angular disposition of approximately 180 degrees, in accordance with an embodiment of the present disclosure. The navigation device mounting joint 45 facilitates coupling of the mounting plate 20 with a navigation device stem 46 by way of a variety of fasteners, as shown by example only. The mounting plate 20 is configured to accept the adapter 43. A pivot arm 47 is pivotally coupled with the navigation device mounting joint 45. The mounting plate 20 is coupled with the pivot arm 47. The camera 307 is safely tucked behind the display device 211 when the navigation arm system is undeployed and locked in place by way of the locking mechanism L. Further, the pivot arm 47 makes a hard stop at an angular position of approximately 180 degrees which is effected by way of locking the handle portion H in the locking mechanism L. As such, the navigation device, such as the camera 307 remains stationary until actually deployed.

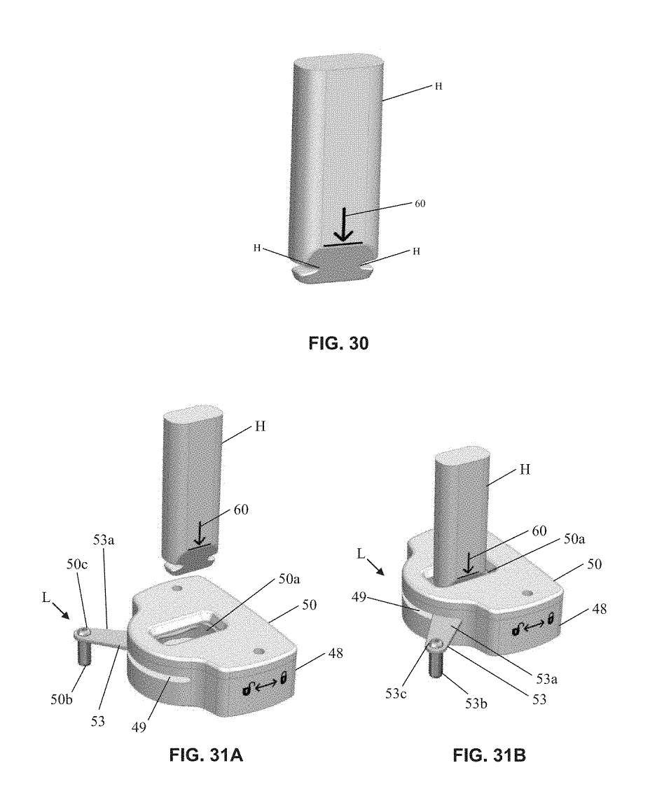

Referring to FIG. 30, this diagram illustrates, in a perspective view of a handle portion H of an arm assembly handle H_{AA}, as comprising a plurality of notches 27 for engaging the cams 51a of the ring lock 51, in accordance with an embodiment of the present disclosure. The handle portion H comprises any configuration that is ergonomically suitable for manual operation, e.g., by a surgeon 201.

Referring to FIG. 31A, this diagram illustrates, in a perspective view, a locking mechanism L, comprising a ring handle 53, interacting with a handle portion H of an arm assembly handle H_{AA}, disposed in a fully unlocked position and ready to accept the handle portion H of an arm assembly handle H_{AA}, in accordance with an embodiment of the present disclosure. The handle portion H further comprises indicia 60, e.g., for representing a direction in which to move the handle H for locking. The cap 50 comprises a slot 50a through which the handle portion H is disposable.

Referring to FIG. 31B, this diagram illustrates, in a perspective view, a locking mechanism L, comprising a ring handle 53, interacting with a handle portion H of an arm assembly handle H_{AA}, disposed in a fully locked position, having accepted the handle portion H, in accordance with an embodiment of the present disclosure. The handle portion H further comprises indicia 60, e.g., for representing a direction in which to move the handle H for locking. The cap 50 comprises a slot 50a through which the handle portion H is disposable.

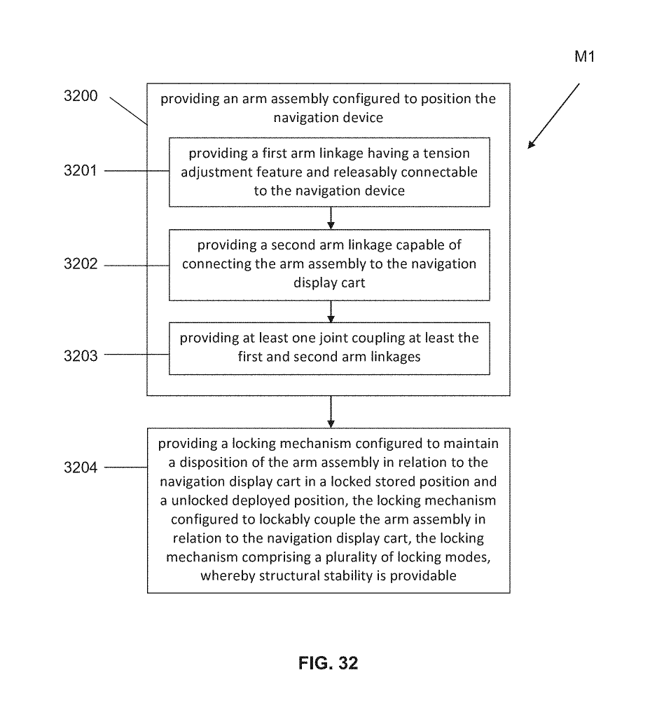

Referring to FIG. 32, this flow diagram illustrates a method M1 of fabricating a navigation arm system S_{ND} for supporting a navigation device, such as the camera 307, of a navigation system 205, capable of coupling with a navigation display cart C_{ND}, the method M1 comprising: providing an arm assembly A_{A} configured to position the navigation device, as indicated by block 3200, providing the arm assembly, as indicated by block 3200, comprising: providing a first arm linkage A₁ having a tension adjustment feature and releasably connectable to the navigation device, as indicated by block 3201; providing a second arm linkage A₂ capable of connecting the arm assembly A_{A} to the navigation display cart C_{ND}, as indicated by block 3202; and providing at least one joint, e.g., joints 32, 31, coupling at least the first and second arm linkages, A₁, A₂, as indicated by block 3203; and providing a locking mechanism L configured to maintain a disposition of the arm assembly A_{A} in relation to the navigation display cart C_{ND} in a locked stored position and a unlocked deployed position, the locking mechanism L configured to lockably couple the arm assembly A_{A} in relation to the navigation display cart C_{ND}, the locking mechanism L comprising a plurality of locking modes, whereby structural stability is providable, as indicated by block 3204, in accordance with an embodiment of the present disclosure.

Still referring to FIG. 32, the method M1 further comprises providing a plurality of mounting brackets, providing the plurality of mounting brackets comprising providing a the navigation device mounting bracket 20, configured to accept the navigation device, such as the camera 307, and providing a cart mounting bracket 46, configured to couple the arm assembly A_{A} in relation to the navigation display cart C. The method M1 further comprises providing an arm assembly handle H_{AA} configured to couple with the navigation device mounting bracket 20, providing the arm assembly handle H_{AA} comprising providing at least one notch 27 and configuring the arm assembly handle H_{AA} to lockably couple with the locking mechanism L and to facilitate positioning of the navigation device, such as the camera 307.

Still referring to FIG. 32, in the method M1, providing the arm assembly, as indicated by block 3200, comprises providing the arm assembly A_{A} as deployable in operation and as retractable during non-operation, providing the locking mechanism L further comprises configuring the locking mechanism L to lockably couple the arm assembly handle H_{AA} in relation to the navigation display cart C_{ND}, whereby a profile of the arm assembly A_{A} is minimized during transportation of the navigation display cart. Providing the locking mechanism L, as indicated by block 3204, further comprises configuring the locking mechanism L to maintain a disposition of the navigation device, such as the camera 307, in relation to the arm assembly A_{A}, providing the first arm linkage A₁, as indicated by block 3201, comprises providing the tension adjustment feature, providing the tension adjustment feature comprising providing a hydraulic piston a piston, such as a hydraulic piston 34, and providing a gas-driven spring 35, whereby the first arm linkage A₁ is configured to counterweight the navigation device, such as the camera 307, and whereby navigation integrity is maintainable.

Still referring to FIG. 32, in the method M1, providing the locking mechanism L, as indicated by block 3204, comprises providing: a base 48 having a slit 49, a cap 50, a lock ring 51, at least one ball-and-compression-spring assembly 52, a ring handle 53, a compression spring 54, a lock tab 55, at least one washer 56, and at least one bolt 57, e.g., at least one shoulder bolt, the base 48 accommodating the cap 50, the lock ring 51, the at least one ball-and-compression-spring assembly 52, the ring handle 53, the compression spring 54, the lock tab 55, the at least one washer 56, and the at least one bolt 57, e.g., at least one shoulder bolt.

Still referring to FIG. 32, in the method M1, providing the ring handle 53 comprises: providing a ring portion 53a operably engageable with the lock ring 51 and the lock tab 55; and providing a ring handle portion 53b coupled with the ring portion 53a and disposable through the slit 49 of the base 48, providing the ring handle 53 comprises configuring the ring handle 53 to actuate the ring lock 51 configured to contemporaneously engage and contemporaneously disengage (a) the at least one cam 51a in relation to at least one notch 27 of the arm assembly handle H_{AA}, whereby the at least one ball-and-compression-spring assembly 52 moves the ball 52a into at least one detent 51c by releasing the spring 52b and (b) the lock tab 55 in relation to a vertical column VC or a spine of the display device cart or navigation display cart C_{ND} by way of the flange 51b being rotated, whereby the compression spring 54 is released and urges the lock tab outward from the base 48 or the locking mechanism L, wherein providing the compression spring 54 comprises configuring the compression spring 54 to urge the lock tab 55 outward in relation to the base 48, and wherein providing lock ring 51 comprises configuring a cam or flange 51b to urge the lock tab 55 inward against the compression spring 54 and in relation to the base 48.

Still referring to FIG. 32, the method M1 further comprises configuring the compression spring 54 to urge the lock tab 55 outboard in relation to the base 48, wherein the cam or flange 51b is configured to urge the lock tab 55 inward against the compression spring 54 and in relation to the base 48. Providing the second arm linkage A₂, as indicated by block 3202, comprises configuring the second arm linkage A₂ to couple the arm assembly A_{A} at a top of a vertical column VC or a spine of the display device cart or navigation display cart C_{ND}. Providing the second arm linkage A₂, as indicated by block 3202, comprises configuring the second arm linkage A₂ for disposition at an obtuse angle, and whereby, when in a deployed position, the arm assembly A_{A} is capable of clearing the navigation display device, such as the display device 211, disposed in relation to the navigation display cart C_{ND}. The obtuse angle comprises a range of at least approximately 120°. Providing the locking mechanism, as indicated by block 3304, comprises configuring the locking mechanism L to couple with a vertical column VC or a spine of the display device cart or navigation display cart C_{ND}.

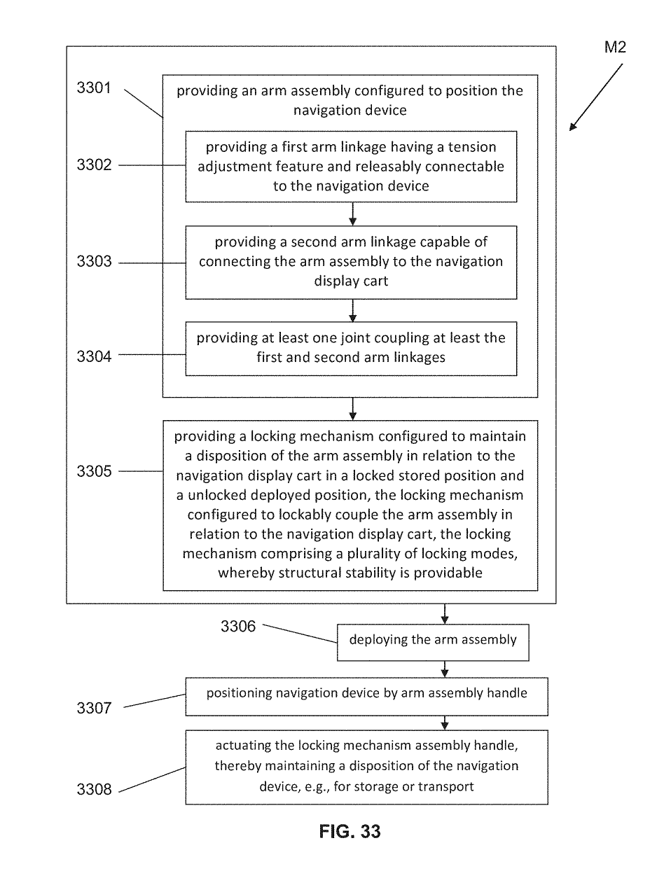

Referring to FIG. 33, this flow diagram illustrates a method M2 of positioning a navigation device, such as a camera 307, by way of a navigation arm system S_{ND}, comprising:

providing the navigation arm system S_{ND} for supporting a navigation device of a navigation system, such as the medical navigation system 205, capable of coupling with a navigation display cart C_{ND}, as indicated by block 3300, providing the system S_{ND}, as indicated by block 3300, comprising: providing an arm assembly A_{A} configured to position the navigation device, as indicated by block 3301, providing the arm assembly A_{A}, as indicated by block 3301, comprising: providing a first arm linkage A₁ having a tension adjustment feature and releasably connectable to the navigation device, as indicated by block 3302; providing a second arm linkage A₂ capable of connecting the arm assembly A_{A} to the navigation display cart C_{ND}, as indicated by block 3303; and providing at least one joint, e.g., joints 32, 31, coupling at least the first and second arm linkages, as indicated by block 3304; and providing a locking mechanism L configured to maintain a disposition of the arm assembly A_{A} in relation to the navigation display cart in a locked stored position and a unlocked deployed position, the locking mechanism configured to lockably couple the arm assembly in relation to the navigation display cart C_{ND}, the locking mechanism L comprising a plurality of locking modes, whereby structural stability is providable, as indicated by block 3305; deploying the arm assembly A_{A}, as indicated by block 3306; positioning the navigation device by way of the arm assembly handle H_{AA}, as indicated by block 3307; and actuating the locking mechanism L, as indicated by block 3308, thereby maintaining a disposition of the navigation device, e.g., for storage or transport, in accordance with an embodiment of the present disclosure.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

### INDUSTRIAL APPLICABILITY

The present disclosure industrially applies to navigation systems. More particularly, the present disclosure industrially applies to navigation devices of navigation systems. Even more particularly, the present disclosure industrially applies to positioning navigation devices of navigation systems.

What is claimed:

1. A navigation arm system for supporting a navigation device of a navigation system, capable of coupling with a navigation display cart, the navigation arm system comprising:
an arm assembly configured to position the navigation device, the arm assembly comprising: a first arm linkage having a tension adjustment feature and releasably connectable to the navigation device; a second arm linkage capable of connecting the arm assembly to the navigation display cart; and at least one joint coupling at least the first and second arm linkages; and
a locking mechanism configured to maintain a disposition of the arm assembly in relation to the navigation display cart in a locked stored position and an unlocked deployed position, the locking mechanism configured to lockably couple the arm assembly in relation to the navigation display cart, the locking mechanism comprising a plurality of locking modes, whereby structural stability is providable.

2. The system of claim 1, further comprising a plurality of mounting brackets, the plurality of mounting brackets comprising a navigation device mounting bracket configured to accept the navigation device and a cart mounting bracket configured to couple the arm assembly in relation to the navigation display cart.

3. The system of claim 2, further comprising an arm assembly handle configured to couple with the navigation device mounting bracket, the arm assembly handle comprising at least one notch and configured to lockably couple with the locking mechanism and to facilitate positioning of the navigation device.

4. The system of claim 1, wherein the arm assembly is deployable in operation and retractable during non-operation.

5. The system of claim 3, wherein the locking mechanism is further configured to lockably couple the arm assembly handle in relation to the navigation display cart, whereby a profile of the arm assembly is minimized during transportation of the navigation display cart.

6. The system of claim 1, wherein the locking mechanism is further configured to maintain disposition of the navigation device in relation to the arm assembly.

7. The system of claim 1, wherein the tension adjustment feature comprises a hydraulic piston and a gas-driven spring, whereby the first arm linkage is configured to counterweight the navigation device, and whereby navigation integrity is maintainable.

8. The system of claim 1, wherein the locking mechanism comprises a base having a slit, a cap, a lock ring, at least one ball-and-compression-spring assembly, a ring handle, a compression spring, a lock tab, at least one washer, and at least one bolt, the base accommodating the cap, the lock ring, the at least one ball-and-compression-spring assembly, the ring handle, the compression spring, the lock tab, the at least one washer, and the at least one shoulder bolt.

9. The system of claim 8,
wherein the ring handle comprises a ring portion and a handle portion,
wherein the lock ring comprises at least one cam, at least one flange, and at least one detent, and
wherein the ring handle is configured to actuate the lock ring for one of contemporaneously engaging and contemporaneously disengaging:
the at least one cam in relation to at least one notch of an arm assembly handle of the arm assembly; and
the lock tab in relation to a vertical column of the navigation display cart.

10. The system of claim 9, wherein the compression spring is configured to urge the lock tab outward in relation to the base, and wherein the cam is configured to urge the lock tab inward against the compression spring and in relation to the base.

11. The system of claim 1, wherein the navigation device comprises an optical camera.

12. The system of claim 1, wherein the second arm linkage is configured to couple the arm assembly with a top portion of a vertical column of the navigation display cart.

13. The system of claim 1, wherein the second arm linkage is configured for disposition thereof in an obtuse angle, and whereby, when in a deployed position, the arm assembly is capable of clearing the navigation display device disposed in relation to the navigation display cart.

14. The system of claim 13, wherein the obtuse angle comprises a range of at least approximately 120 degrees.

15. The system of claim 1, wherein the locking mechanism is configured to couple with a vertical column of the navigation display cart.

16. A method of fabricating a navigation arm system for supporting a navigation device of a navigation system, capable of coupling with a navigation display cart, the method comprising:
providing an arm assembly configured to position the navigation device, providing the arm assembly comprising: providing a first arm linkage having a tension adjustment feature and releasably connectable to the navigation device; providing a second arm linkage capable of connecting the arm assembly to the navigation display cart; and providing at least one joint coupling at least the first and second arm linkages; and
providing a locking mechanism configured to maintain a disposition of the arm assembly in relation to the navigation display cart in a locked stored position and a unlocked deployed position, the locking mechanism configured to lockably couple the arm assembly in relation to the navigation display cart, the locking mechanism comprising a plurality of locking modes, whereby structural stability is providable.

17. The method of claim 16, further comprising providing a plurality of mounting brackets, providing the plurality of mounting brackets comprising providing a navigation device mounting bracket configured to accept the navigation device and providing a cart mounting bracket configured to couple the arm assembly in relation to the navigation display cart.

18. The method of claim 17, further comprising providing an arm assembly handle configured to couple with the navigation device mounting bracket, providing the arm assembly handle comprising providing at least one notch and configuring the arm assembly handle to lockably couple with the locking mechanism and to facilitate positioning of the navigation device.

19. The method of claim 16, wherein providing the arm assembly comprises providing the arm assembly as deployable in operation and as retractable during non-operation.

20. The method of claim 18, wherein providing the locking mechanism further comprises configuring the locking mechanism to lockably couple the arm assembly handle in relation to the navigation display cart, whereby a profile of the arm assembly is minimized during transportation of the navigation display cart.

21. The method of claim 16, wherein providing the locking mechanism further comprises configuring the locking mechanism to maintain disposition of the navigation device in relation to the arm assembly.

22. The method of claim 16, wherein providing the first arm linkage comprises providing the tension adjustment feature, providing the tension adjustment feature comprising providing a hydraulic piston and providing a gas-driven spring, whereby the first arm linkage is configured to counterweight the navigation device, and whereby navigation integrity is maintainable.

23. The method of claim 16, wherein providing the locking mechanism comprises providing: a base having a slit, a cap, a lock ring, at least one ball-and-compression-spring assembly, providing a ring handle, a compression spring, a lock tab, at least one washer, and at least one bolt, the base accommodating the cap, the lock ring, the at least one ball-and-compression-spring assembly, the ring handle, the compression spring, the lock tab, the at least one washer, and the at least one shoulder bolt.

24. The method of claim 23,
wherein providing the ring handle comprises providing a ring portion and a handle portion,
wherein providing the lock ring comprises providing at least one cam, at least one flange, and at least one detent, and
wherein the providing ring handle comprises configuring the ring handle to actuate the lock ring for one of contemporaneously engaging and contemporaneously disengaging:
the at least one cam in relation to at least one notch of an arm assembly handle of the arm assembly; and
the lock tab in relation to a vertical column of the navigation display cart.

25. The method of claim 24, wherein providing the compression spring comprises configuring the compression spring to urge the lock tab outward in relation to the base, and wherein the cam is configured to urge the lock tab inward against the compression spring and in relation to the base.

26. The method of claim 16, wherein the navigation device comprises an optical camera.

27. The method of claim 16, wherein providing the second arm linkage comprises configuring the second arm linkage to couple the arm assembly at a top of a vertical column of the navigation display cart.

28. The method of claim 16, wherein providing the second arm linkage comprises configuring the second arm linkage for disposition in an obtuse angle, and whereby, when in a deployed position, the arm assembly is capable of clearing the navigation display device disposed in relation to the navigation display cart.

29. The method of claim 28, wherein the obtuse angle comprises a range of at least approximately 120 degrees.

30. The method of claim 16, wherein providing the locking mechanism comprises configuring the locking mechanism to couple with a vertical column of the navigation display cart.

31. A method of positioning a navigation device by way of a navigation arm system, comprising:
providing the navigation arm system for supporting a navigation device of a navigation system, capable of coupling with a navigation display cart, providing the system comprising:
providing an arm assembly configured to position the navigation device, providing the arm assembly comprising: providing a first arm linkage having a tension adjustment feature and releasably connectable to the navigation device; providing a second arm linkage capable of connecting the arm assembly to the navigation display cart; and providing at least one joint coupling at least the first and second arm linkages; and
providing a locking mechanism configured to maintain disposition of the arm assembly in relation to the navigation display cart in a locked stored position and a unlocked deployed position, the locking mechanism configured to lockably couple the arm assembly in relation to the navigation display cart, the locking mechanism comprising a plurality of locking modes, whereby structural stability is providable;
deploying the arm assembly;
positioning the navigation device by way of the arm assembly,
thereby maintaining disposition of the navigation device.
